(19) United States
(12) Patent Application Publication
(10) Pub. No.: US 2026/0256790 A1
(43) Pub. Date: Sep. 3, 2026

(54) DEBIO-0123 IN COMBINATION WITH TEMOZOLOMIDE AND RADIOTHERAPY FOR USE IN TREATING GLIOMA

(71) Applicant: Debiopharm International S.A., Lausanne (CH)

(72) Inventors: Anne Vaslin Chessex, Prilly (CH); Lars Damstrup, Hillerod (DK); Anne Bellon, Pully (CH); Esteban Rodrigo Imedio, Madrid (ES); Victor Rodriguez Freixinos, Pully (CH); Luke Piggott, Pully (CH)

(73) Assignee: Debiopharm International S.A., Lausanne (CH)

(21) Appl. No.: 19/155,715

(22) PCT Filed: Feb. 12, 2024
(86) PCT No.: PCT/EP2024/053449

§ 371 Date: Aug. 12, 2025

(30) Foreign Application Priority Data
Feb. 13, 2023 (EP) 23156300.8
Mar. 2, 2023 (EP) 23159653.7

(51) Int. Cl. A61K 31/519; A61K 31/495; A61N 5/10; A61P 35/00

(52) U.S. Cl. CPC A61K 31/519; A61K 31/495; A61N 5/10; A61P 35/00

(57) ABSTRACT

Methods of treating glioma using a WEE1 inhibitor are disclosed.

### FIELD OF THE INVENTION

The present invention generally relates to the treatment of glioma. More specifically, the present invention relates to the use of a specific WEE1 inhibitor to treat glioma in patients in need thereof.

### BACKGROUND

Worldwide, it is estimated that over 300′000 people were diagnosed with a primary brain tumor in 2020. Brain tumors account for 85% to 90% of all primary central nervous system (CNS) tumors. Pursuant to the World Health Organization 2021 categorization (WHO 2021), CNS tumors comprise many categories, including one called “gliomas, glioneuronal tumors, and neuronal tumors”. These tumors can be further classified by subtypes, as well as by grades, for example based on an integrated histological and molecular grading, from 3 to 4, with grade 4 being the most aggressive.

High-grade diffuse glioma (also referred to as highly malignant glioma) accounts for more than half of malignant primary brain tumors. Approximately 60-70% of all malignant gliomas are glioblastoma (GBM). GBM is the most common malignant primary brain tumor in adults and represents a major cause of morbidity and mortality. Of all GBMs, primary GBM constitutes approximately 90%. In contrast to primary GBM (95% associated with Isocitrate dehydrogenase (IDH)-wildtype status), IDH1 gene mutations are common for secondary GBM and the tumors formerly known as anaplastic astrocytoma, which are now classified as astrocytoma IDH-mutant Grade 3 tumors as per the WHO 2021 classification. Across grades, the presence of an IDH mutation generally identifies a group of tumors with a more favorable prognosis than their IDH-wildtype counterpart.

Following surgical resection, the widely accepted standard of care (SOC) for patients with newly diagnosed GBM who are in good general condition consists of radiation therapy (RT) with concurrent temozolomide (TMZ), generally followed by maintenance of TMZ (Stupp R, et al. 2005. ‘Radiotherapy plus Concomitant and Adjuvant Temozolomide for Glioblastoma’, New England Journal of Medicine, 352 (10): 987-96).

Additional FDA-approved treatment options have been incorporated for newly diagnosed, high-grade glioma: direct intracranial application of chemotherapy with biodegradable carmustine-impregnated wafers as an adjunct to surgery and the addition of alternating, electric tumor treating fields (TTFields) therapy during maintenance of TMZ treatment (Stupp R, et al. 2017. ‘Effect of tumor-treating fields plus maintenance temozolomide vs. maintenance temozolomide alone on survival in patients with glioblastoma: a randomized clinical trial’, JAMA, 318: 2306-16). Unfortunately, the addition of immunotherapy or systemic targeted therapies, including bevacizumab, to the SOC for patients with newly diagnosed high-grade glioma has not been shown to improve overall survival (OS). Despite additional therapies, the prognosis for patients with newly diagnosed high-grade glioma remains poor. Nearly all recur after 6-9 months following first line therapy, with a 5-year OS of less than 10%.

The management of recurrent GBM also continues to be challenging. There is no established second-line therapy, and effective treatment options are limited. Therapies may include repeated neurosurgical resection, re-irradiation, TTFields, locally-delivered chemotherapy with biodegradable carmustine-impregnated wafers, bevacizumab, systemic cytotoxic chemotherapy (such as re-exposure to TMZ, treatment with nitrosureas, or combinations of carboplatin and etoposide), and supportive care. However, their effectiveness remains limited, with response rates of ≤15%, progression free survival at 6 months (PFS-6) rates typically ranging from 9% to 21%, and median OS of ≤30 weeks.

As an example, since 2009, bevacizumab has been an FDA-approved treatment option for the treatment of recurrent GBM (see e.g. Friedman H S, et al. 2009. ‘Bevacizumab alone and in combination with irinotecan in recurrent glioblastoma’, J Clin Oncol, 27: 4733-40). However, bevacizumab prolongs PFS but not OS in patients with recurrent GBM. More generally, in patients who experience relapse, no clinical trials have demonstrated an improvement in OS with second-line systemic therapies (including targeted therapy or immunotherapy, see e.g. Lombardi et al. 2020. ‘A New Landscape for Systemic Pharmacotherapy of Recurrent Glioblastoma?’, Cancers, 12: 3775).

Given the poor outcomes with current therapies, there is an urgent unmet medical need to develop novel treatment interventions to improve clinical outcomes and/or quality of life (QoL) for patients suffering from newly diagnosed or recurrent glioma, and particularly from high-grade glioma.

### SUMMARY OF THE INVENTION

The present invention relates to a compound of formula (I)

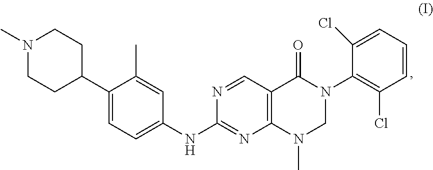 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof for use in, or for use in the preparation of a medicament for, treating glioma in a patient in need thereof.

The present invention also relates to a method for treatment of glioma in a patient in need thereof using a compound of formula (I) or a pharmaceutically acceptable salt thereof.

In some aspects of the uses or methods according to the present invention, the compound of formula (I) is used in combination with temozolomide (TMZ) and/or radiotherapy. The compound of formula (I) may also be used in combination with both temozolomide and radiotherapy.

In some aspects of the uses or methods according to the present invention, the glioma is glioblastoma, astrocytoma or oligodendroglioma.

In some aspects of the uses or methods according to the present invention, the glioma is a high-grade glioma (Grade 3 or 4).

In some aspects of the uses or methods according to the present invention, the compound of formula (I) is administered orally
at an intermittent schedule, preferably selected from any one of (i) to (vi):
   i. on days 1 to 5, 8 to 10 and 15 to 17 of a 28-day cycle,
   ii. on days 1 to 5, 8 to 12 and 15 to 19 of a 28-day cycle,
   iii. on days 1 to 5, 8 to 12, 15 to 19 and 22-26 of a 28-day cycle,
   iv. on days 1 to 3 of each week, for 6 or 7 weeks or 42-49 days,
   v. on days 1 to 5 of each week, for 6 or 7 weeks or 42-49 days, or
   vi. on days 1 to 4 of each week, for 6 or 7 weeks or 42-49 days, or at a continuous schedule, preferably
   vii. daily for 6 weeks, daily for 7 weeks, or daily for 42 to 49 days.

The administration on days 1 to 5, 8 to 10 and 15 to 17 of a 28-day cycle, on days 1 to 5, 8 to 12 and 15 to 19 of a 28-day cycle, or on days 1 to 5, 8 to 12, 15 to 19 and 22-26 of a 28-day cycle may be preferred when the glioma has progressed or recurred after initial or prior glioma treatment. The administration on days 1 to 3 of each week, for 6 or 7 weeks or 42-49 days, on days 1 to 5 of each week, for 6 or 7 weeks or 42-49 days, on days 1 to 4 of each week, for 6 or 7 weeks or 42-49 days, or daily for 6 weeks, daily for 7 weeks, or daily for 42 to 49 days may be preferred if the glioma has been newly diagnosed.

In some aspects of the uses or methods according to the present invention, the compound of formula (I) is administered over 1, 2, 3, 4, 5, 6 or more 28-day cycles. Typically, there are no breaks between any consecutive cycles, i.e. the day following a prior 28-day cycle may be the first day of the consecutive cycle.

In some aspects of the uses or methods according to the present invention, the compound of formula (I) is administered
i. at a dose ranging from about 30 to about 1000 mg per treatment day, preferably about 30 to about 720 mg per treatment day, and in some embodiments more preferably about 90 to about 720 mg per treatment day or about 150 to about 720 mg per treatment day, and/or
ii. as a single dose on a treatment day, and/or
iii. at approximately the same time on each treatment day such as at the same time ±about 60 min, preferably ±60 min.

In some aspects of the uses or methods according to the present invention, the compound of formula (I) is used in combination with temozolomide, and temozolomide is administered
i. at a dose of 50-300 mg/m²/day, preferably 50-250 mg/m²/day, further preferably at a dose of 50-200 mg/m²/day on days 1 to 5 of a 28-day cycle,
ii. at a dose of 150 mg/m²/day on days 1 to 5 of a 28-day cycle,
iii. at a dose of 200 mg/m²/day on days 1 to 5 of a 28-day cycle,
iv. at a dose of 50-300 mg/m²/day, preferably 50-250 mg/m²/day, further preferably 50-200 mg/m²/day, daily for 6 weeks, daily for 7 weeks or daily for 42 to 49 days, or
v. at a dose of 75 mg/m²/day, daily for 6 weeks, daily for 7 weeks or daily for 42 to 49 days.

The administration of TMZ on days 1 to 5 of a 28-day cycle may be preferred when the glioma has progressed or recurred after initial or prior glioma treatment, preferably 1 or 2 prior treatment lines, including typically temozolomide (TMZ)-based chemoradiotherapy as first-line treatment. The administration of TMZ daily for 6 weeks, daily for 7 weeks, or daily for 42 to 49 days may be preferred if the glioma has been newly diagnosed.

In some aspects of the uses or methods according to the present invention, the compound of formula (I) is used in combination with temozolomide, and on a WEE1 inhibitor treatment day, if temozolomide is administered on said WEE1 inhibitor treatment day, administration of the compound of formula (I) precedes administration of temozolomide, optionally by a period of about 30 min to 4 h.

In some aspects of the uses or methods according to the present invention, the compound of formula (I) is used in combination with temozolomide, and on a WEE1 inhibitor treatment day, if temozolomide is administered on said WEE1 inhibitor treatment day, temozolomide administration is concomitant to administration of the compound of formula (I).

In some aspects of the uses or methods according to the present invention, said compound of formula (I) is used in combination with temozolomide and radiotherapy. Preferably, the compound of formula (I) is used in combination with temozolomide and radiotherapy for the treatment of glioma that is newly diagnosed.

In some aspects of the uses or methods according to the present invention, the compound of formula (I) is administered daily for 6 weeks, daily for 7 weeks or daily for 42 to 49 days, and temozolomide is administered at a dose of 50-300 mg/m²/day, preferably 75 mg/m²/day, daily for 6 weeks, daily for 7 weeks, or daily for 42 to 49 days. Preferably, this schedule is used for the treatment of glioma that is newly diagnosed.

In some aspects of the uses or methods according to the present invention, the compound of formula (I) is administered
i. on days 1 to 3 of each week, for 6 or 7 weeks or 42-49 days,
ii. on days 1 to 5 of each week, for 6 or 7 weeks or 42-49 days,
iii. on days 1 to 4 of each week, for 6 or 7 weeks or 42-49 days, or
iv. daily for 6 or 7 weeks or 42-49 days
   and temozolomide is administered at a dose of 50-300 mg/m²/day, preferably 75 mg/m²/day, daily for 6 weeks, daily for 7 weeks, or daily for 42 to 49 days.

Preferably, this schedule is used for the treatment of glioma that is newly diagnosed.

In some aspects of the uses or methods according to the present invention, the radiotherapy is fractionated radiotherapy and is administered
i. in a regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks or 42-49 days,
ii. in a regimen of 2 Gy per fraction daily, 5 consecutive days per week for a total of 60 Gy, or
iii. at a total dose of 60 Gy over 30 fractions, over a period of 6 weeks, 7 weeks or 42 to 49 days.

In some aspects of the uses or methods according to the present invention, the glioma is newly diagnosed. In these aspects, the compound of formula (I) is preferably administered orally
i. on days 1 to 3 of each week, for 6 or 7 weeks or 42-49 days,
ii. on days 1 to 5 of each week, for 6 or 7 weeks or 42-49 days,
iii. on days 1 to 4 of each week, for 6 or 7 weeks or 42-49 days, or
iv. daily for 6 or 7 weeks or 42-49 days, and/or
   the compound of formula (I) is preferably used in combination with temozolomide, and temozolomide is administered
i. at a dose of 50-300 mg/m²/day, preferably 50-250 mg/m²/day, further preferably 50-200 mg/m²/day, daily for 6 weeks, daily for 7 weeks or daily for 42 to 49 days, or
ii. at a dose of 75 mg/m²/day, daily for 6 weeks, daily for 7 weeks or daily for 42 to 49 days, and/or
   the compound of formula (I) is preferably used in combination with temozolomide and radiotherapy, optionally wherein the compound of formula (I) is administered as specified above, and wherein temozolomide is administered at a dose of 50-300 mg/m²/day, preferably 75 mg/m²/day, daily for 6 weeks, daily for 7 weeks, or daily for 42 to 49 days; and/or optionally wherein the radiotherapy is fractionated radiotherapy and is administered
i. in a regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks or 42-49 days,
ii. in a regimen of 2 Gy per fraction daily, 5 consecutive days per week for a total of 60 Gy, or
iii. at a total dose of 60 Gy over 30 fractions, over a period of 6 weeks, 7 weeks or 42 to 49 days.

In some aspects of the uses or methods according to the present invention, the glioma has progressed or recurred after initial or prior glioma treatment. In these aspects, the compound of formula (I) is preferably administered orally
i. on days 1 to 5, 8 to 10 and 15 to 17 of a 28-day cycle,
ii. on days 1 to 5, 8 to 12 and 15 to 19 of a 28-day cycle, or
iii. on days 1 to 5, 8 to 12, 15 to 19 and 22-26 of a 28-day cycle,
   wherein the compound of formula (I) may be administered over 1, 2, 3, 4, 5, 6 or more 28-day cycles; and/or the compound of formula (I) is used in combination with temozolomide, and temozolomide is administered
i. at a dose of 50-300 mg/m²/day, preferably 50-250 mg/m²/day, further preferably at a dose of 50-200 mg/m²/day on days 1 to 5 of a 28-day cycle,
ii. at a dose of 150 mg/m²/day on days 1 to 5 of a 28-day cycle, or
iii. at a dose of 200 mg/m²/day on days 1 to 5 of a 28-day cycle.

In some aspects of the uses or methods according to the present invention, the initial or prior glioma treatment comprises radiotherapy administration and/or TMZ administration.

In some aspects of the uses or methods according to the present invention, the glioma is glioblastoma.

In some aspects of the uses or methods according to the present invention, the glioma is (IDH)-wildtype as per WHO 2021.

In some aspects of the uses or methods according to the present invention, the glioma is astrocytoma.

In some aspects of the uses or methods according to the present invention, the glioma is IDH-mutant as per WHO 2021.

### BRIEF DESCRIPTION OF THE DRAWINGS

FIG. 1 shows graphs representing the plasma and brain concentrations in healthy mice measured on day 4, following repeated dose administration of 30 mg/kg qd of Compound of formula (I) (FIG. 1(A)), 30 mg/kg bid of AZD1775 (FIG. 1(B)) and 80 mg/kg qd of Zn-c3 (FIG. 1(C)), as described in Example 1.

FIG. 2 is a graph showing the total plasma concentration, total brain concentration, unbound CSF concentration and calculated unbound plasma concentration of Compound of formula (I) over time, following single dose administration of 15 mg/kg of Compound of formula (I) in healthy rats, as described in Example 2.

FIG. 3 shows graphs representing the in vitro efficacy of Compound of formula (I) [CoF (I)], at various doses, in combination with radiotherapy (RT) in T98G (FIG. 3A), LN18 (FIG. 3B), SNB-19 (FIG. 3C) and U251 (FIG. 3D) GBM cell lines, as described in Example 4.

FIG. 4 shows graphs representing in vivo efficacy of compound of formula (I) [CoF (I)] as a monotherapy in the U87-MG-luc GBM cell line, implanted in mice either subcutaneously and treated with Compound of formula (I) at 30 mg/kg or 60 mg/kg (FIG. 4A, tumor volume) or cerebrally and treated with Compound of formula (I) at 30 mg/kg (FIG. 4B, relative BLI), as described in Example 5.

FIG. 5 is a graph representing in vivo efficacy of compound of formula (I) [CoF (I)] in combination with TMZ in the U87-MG-luc GBM cell line implanted intracranially in mice as described in Example 6, showing the relative BLI curves at doses of 3 mg/kg TMZ, 10 mg/kg CoF (I), and combination of 3 mg/kg TMZ+10 mg/kg CoF (I).

FIG. 6 is a graph showing probability of survival of mice harboring GL261 intracranial tumors when treated with TMZ (3 mg/kg) as a monotherapy, compound of formula (I) (30 mg/kg) [CoF (I)] as a monotherapy or a combination of both, as described in Example 7.

FIG. 7 represents the in vitro efficacy of compound of formula (I) as a monotherapy in ATRX deficient SF188 GBM cell lines, as described in Example 8: cell viability assays (FIGS. 7A and 7B) and colony formation assay (FIG. 7C).

FIG. 8 is a Clinical Study diagram for Phase 1 Arm A as described in Example 9.

FIG. 9 is a Clinical Study diagram for Phase 1 Arm B as described in Example 9.

FIG. 10 is a Clinical Study diagram for Phase 2 Experimental Arm as described in Example 9.

### DETAILED DESCRIPTION OF THE INVENTION

The present invention relates to the use of a compound of formula (I), or a pharmaceutically acceptable salt thereof, in the treatment of glioma in patients in need thereof. The compound of formula (I) is a WEE1 inhibitor. In some embodiments, an N-oxide or N-oxide derivative of the compound of formula (I) may be used.

It has been found that the compound of formula (I) shows good ability to cross the blood-brain barrier, in healthy mice, rats and monkeys. Furthermore, it has been found that the compound of formula (I) as single agent or in combination with TMZ shows antitumor activity and good tolerability in mouse GBM xenograft models. Additionally, it has been found that the compound of formula (I) shows radiosensitizing effects for multiple cell lines, including GBM cell lines.

### Definitions

So that the invention may be more readily understood, certain terms are specifically defined below. Unless explicitly defined elsewhere in this document, all other technical and scientific terms used herein have the meaning that would be commonly understood by one of ordinary skill in the relevant art.

As used herein, including in the appended claims, the singular forms of words such as “a”, “an”, and “the”, include their corresponding plural references unless the context clearly indicates otherwise.

It is understood that wherever embodiments are described herein with the language “comprising,” otherwise analogous embodiments described in terms of “consisting of” and/or “consisting essentially of” are also provided.

The term “and/or” as used herein in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, a “WEE1 inhibitor” refers to a compound that inhibits the activity of the WEE1 kinase, for example with an IC50 of <10 nM in an ADP-GLO kinase assay or an IC50 of <100 nM in an enzyme profiling assay. The compound of formula (I) herein is an example of a WEE1 inhibitor.

In some aspects, a “brain penetrant” WEE1 inhibitor refers to a WEE1 inhibitor that is able to cross the blood brain barrier (whether impaired or not) and to distribute into the brain. In some aspects, the ability of a compound to cross the blood brain barrier and distribute into the brain is reflected by its brain to plasma concentration ratio exceeding a certain threshold. For example, compound of formula (I) may exhibit a better ability to cross the blood brain barrier and distribute into the brain than AZD1775 and Zn-c3, as evidenced by its higher brain to plasma concentration ratios. Compound of formula (I) may thus be referred to as a brain penetrant WEE1 inhibitor.

As used herein when referring to compound of formula (I), the expression “pharmaceutically acceptable salt” refers to salts prepared from pharmaceutically acceptable non-toxic acids including inorganic or organic acids. For example, acceptable salts derived from acids such as quaternary salt, acetate, carbonate, carbamate, sulfonate, strong inorganic acids and the like. In general, pharmaceutically acceptable salts may be used for modifying the solubility or hydrolysis characteristics of a compound, or in sustained release formulations. It will be understood that, as used herein, references to the compound of formula (I) are meant to also include the pharmaceutically acceptable salts unless stated otherwise.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, administration “in combination with” one or more further therapeutic agents includes simultaneous (concurrent) and consecutive (separate) administration in any order.

As used herein, terms such as “treating” or “treatment” or “to treat” or “alleviating” or “to alleviate” refer to therapeutic measures that cure, slow down, lessen symptoms of, and/or halt or reverse progression or severity of a diagnosed pathologic condition, disorder or disease. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size or burden; inhibition of or an absence of cancer cell infiltration into peripheral organs; inhibition of or an absence of tumor metastasis; inhibition of or an absence of tumor growth; relief of one or more symptoms associated with the specific cancer; reduction in corticosteroid dose need; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state; as well as increased chances to have an increased Overall Response (OR), including a complete response (CR) or a partial response (PR), increased chances to have the disease under control (e.g. CR, PR, stable disease SD), to live longer without progression, and without disease, to live longer, decreased risk to have a progressive disease (PD) and to increase the time until progression. Collectively for groups or populations of patients, successful treatment may result in endpoints such as increased Overall Response Rate (ORR), Best Overall Response (BOR), Duration of Response (DOR), Disease Control Rate (DCR), progression-free survival (PFS), overall survival (OS), time to progression (TTP) or any combination thereof.

As used herein, the term “glioma” refers to a type of primary brain tumor that originates from the glial cells. It includes, for example, glioblastoma, astrocytoma and oligodendroglioma. Glioma may, for example, be classified according to the 5th edition of the World Health Organization (WHO) classification of Central Nervous System (CNS) tumors, 2021, hereafter referred to as WHO 2021 (Louis D N, et al. 2021. ‘The 2021 WHO Classification of Tumors of the Central Nervous System: A summary’, Neuro Oncol, 23: 1231-51). In some embodiments, the WHO classification may evolve in the future and (a) new version(s) may be released and considered.

In some aspects, when referring to a glioma, the WHO 2021 refers to an integrated histological and molecular grading of the tumor. Grades 1 and 2 are generally considered “low grade”, and grades 3 and 4 are generally considered “high grade”.

As used herein, the term “glioma treatment” refers to any treatment or therapy approved by at least one health authority such as FDA or EMA, or under clinical trial investigation to treat glioma. “Initial glioma treatment” refers to the first glioma treatment used for a given patient diagnosed with glioma, including but not limited to surgical resection, radiation therapy (RT) and/or temozolomide (TMZ). “Prior” or “previous glioma treatment” refers to one or more glioma treatment used for a given patient diagnosed with glioma before the methods according to the present invention.

As used herein, the term “glioblastoma”, “glioblastoma multiforme” or “GBM” as per WHO 2021, may be referred to as isocitrate dehydrogenase (IDH)-wildtype diffuse astrocytic Grade 4 tumors.

As used herein, the term “diffuse glioma” is used to encompass a variety of poorly marginated infiltrating tumors of the central nervous system, which histologically appear similar to glial cells, specifically astrocytes and oligodendrocytes. These range in biological behavior from very indolent to extremely aggressive and this is reflected in grading that ranges from grade 1 to grade 4. For clarity, the term diffuse glioma does not include circumscribed astrocytic tumors.

As used herein, the term “astrocytoma” may be referred to as IDH-mutant glioma. For example, as per WHO 2021, IDH-mutant astrocytomas are classified into three WHO grades: astrocytoma, IDH-mutant, WHO grade 2; astrocytoma, IDH-mutant, WHO grade 3 (instead of anaplastic astrocytoma, IDH-mutant, WHO grade 3); and astrocytoma, IDH-mutant, WHO grade 4 (replacing the former term ‘glioblastoma, IDH-mutant, WHO grade 4’).

In some aspects, when referring to a glioma, the term “ATRX-deficient” or “ATRX deficiency” means that the alpha-thalassemia/mental retardation syndrome X-linked (ATRX) gene in the glioma presents alterations such as nonsense or frameshift mutations resulting in a truncated protein and/or decrease or loss of protein expression (often consequent on gene alteration). Such gene alterations may be detected by known methods, including for example whole exome sequencing. Gene expression (or decrease or loss thereof) may be detected by known methods, including for example mass spectrometry proteomics, immunohistochemistry, western blot, for protein levels and/or by measuring mRNA expression level using whole transcriptome sequencing or microarray technology.

In some aspects when referring to a glioma, the “isocitrate dehydrogenase (IDH) status” or “IDH status” or “IDH mutational status” refers to whether or not the isocitrate dehydrogenase enzyme (IDH) genes in the glioma contain mutations. In humans, IDH1, IDH2, and IDH3 genes express three isoforms of the IDH enzyme, which all have significant functions in metabolic reactions. IDH1 is found in the cytoplasm and peroxisome, while IDH2 and IDH3 are in the mitochondrial matrix. The mutations observed in IDH enzymes in gliomas are mainly in cytosolic IDH1 and mitochondrial IDH2, most frequently at codons R132 and R172, respectively. The “IDH status” in glioma generally refers to IDH1 or IDH2, sometimes also referred to as “IDH1/2 status”. In the absence of mutations altering the enzyme activity, the IDH status is said to be “wildtype”. If one or more mutations altering the enzyme activity are present, the IDH status is said to be “IDH mutated” or “IDH mutant”. Mutations in the IDH genes may be detected by known methods, including for example the polymerase chain reaction (PCR) reaction, which may be performed based on Formalin-Fixed, Paraffin-Embedded (FFPE) Glioma Tissues. In some instances, in addition to PCR, immunohistochemical negativity for IDH1 R132H may be used for the classification as IDH-wild-type glioblastoma.

In some aspects when referring to a glioma, the “MGMT status” refers to the promoter methylation status of the gene encoding for the repair enzyme 06-methylguanine-DNA methyltransferase (MGMT), namely whether such promoter is methylated or unmethylated. Possible methods for detecting MGMT status include, without being limited to, non-quantitative methylation-specific polymerase chain reaction (MSP) or quantitative MSP (qMSP), pyrosequencing or methylation-sensitive multiplex ligation-dependent probe amplification (MS-MPLA).

In some aspects when referring to a glioma, the “1p/19q status” refers to whether or not the short arm of chromosome 1 (1p) and the long arm of chromosome 19 (19q) are deleted. Complete deletion of both the short arm of chromosome 1 (1p) and the long arm of chromosome 19 (19q) (1p/19q co-deletion or co-deleted) is generally the molecular genetic signature of oligodendroglioma, a subtype of primary brain tumours accounting for approximately ten to fifteen percent of all diffuse gliomas in adults. In the absence of 1p/19q co-deletion, the status is generally considered “1p/19q intact”. 1p/19q co-deletion in glioma may be tested by known methods such as fluorescent in situ hybridisation (FISH), using dualfluorescent-labelled DNA probes to detect 1p and 19q loci within the interphase nuclei of individual glioma cells from FFPE glioma tissue sections transcribed on to unstained slides; changes in the 1p and 19q probe signals compared with controls are used to determine the presence of 1p/19q-co-deletion. Polymerase chain reaction-based microsatellite analysis—that allows detection of loss of heterozygosity (LOH) at selected loci, and next-generation sequencing—NGS-based methods can also be used to assess 1p/19q status.

In some aspects, when referring to a glioma, the “CDKN2A/B status” refers to whether or not the cyclin-dependent kinase inhibitors 2A and/or 2B (CDKN2A/B) gene is altered, particularly whether there is an homozygous deletion of CDKN2A and CDKN2B. CDKN2A/B are driver tumor suppressor genes, which regulate cell cycle progress. The loss of CDKN2A is believed to promote glioma formation and tumor metastasis, and CDKN2A/B homozygous deletion has been identified as an indicator of poor prognosis, particularly in astrocytoma. In the absence of a homozygous deletion of CDKN2A/B, the glioma may be said to retain CDKN2A/B. A potential alteration of CDKN2A/B may be detected with known methods, such as FISH, qPCR or NGS-based methods.

In some aspects, when referring to a glioma, the “TERT promoter mutation status” or TERTp mutation status refers to whether certain mutations are present in the promoter of the TERT gene. TERTp mutations occur in 51% of all glioma grades. TERTp mutations may be identified using known methods that may be based on molecular characterization of tumor DNA, for example using Sanger sequencing based on tumor DNA sequencing, Droplet Digital PCR (ddPCR), mass-spectrometry-based tests, or next-generation sequencing (NGS).

In some aspects, when referring to a glioma, the “EGFR amplification status” refers to whether or not the Epidermal growth factor receptor (EGFR) gene is amplified (EGFRamp). EGFR amplification may be detected with known methods, such as by Fluorescence In-Situ Hybridization (FISH), Chromosomal Microarray or Real-Time Quantitative PCR.

In some aspects, when referring to a glioma, the “chromosome 7/10 status” refers to whether or not the glioma presents a combination of gain of chromosome 7 (including the EGFR gene) and a loss of chromosome 10 (7+/10−). The chromosome 7/10 status may be assessed using known methods, such as FISH, NGS, Chromosomal Microarray or Multiplex Ligation-dependent Probe Amplification (MLPA).

In absence of microvascular proliferation and/or necrosis in a diffuse glioma, EGFR amplification, the combination of gain of chromosome 7 and loss of chromosome 10 (7+/10−), and TERT promoter mutation (pTERTmut) are alterations frequently observed in adult, and in WHO 2021 are defined as molecular characteristics of IDH-wild-type glioblastoma.

In some aspects, when referring to a glioma, the “H3.3 G34 status” or “H3 K27M status” refers to whether or not the glioma presents histone H3.3 G34 mutations or a loss of nuclear K27-trimethylated histone H3 (H3K27me3), for example due to H3K27 mutation. A glioma may thus be H3.3 G34 wild-type, H3.3 G34 mutant, such as H3.3 G34R/V mutant, or H3 K27M mutant, or may have a loss of H3K27me3. H3.3 G34 mutations (such as H3.3 G34RN) or H3 K27M mutations may be assessed by known methods such as immunohistochemistry or DNA sequencing to identify H3.3 G34-mutant diffuse hemispheric glioma or diffuse midline glioma, H3 K27M-mutant WHO grade 4 respectively. In a glioblastoma or GBM, the H3.3 G34 status is wild type.

As used herein when referring to glioma or to a patient suffering therefrom, the terms “relapse”, “to relapse”, “recurrence”, “to recur”, “recurrent” mean a worsening of the disease and/or of the signs and symptoms of the disease after a period of improvement, stabilization or disease absence.

As used herein when referring to glioma or to a patient suffering therefrom, the terms “progression”, “to progress” or “progressive” means when the cancer becomes worse, either due to existing lesions that are growing and/or due to appearance of new lesions.

As used herein, the term “WEE1 inhibitor treatment day” refers to a day on which the compound of formula (I) is administered according to the methods of the present invention.

As used herein, the term “therapeutically effective amount” of a given drug when used in monotherapy, such as a “WEE1 therapeutically effective amount” or a “TMZ therapeutically effective amount”, refers to an amount of the drug effective to “treat” a disease or disorder in a subject or patient, whilst keeping an acceptable safety profile. In some aspects, when administering WEE1 and TMZ in combination (optionally further with radiotherapy), their respective therapeutically effective amounts may be referred to as the “WEE1 combination therapeutically effective amount” and the “TMZ combination therapeutically effective amount”. The “WEE1 therapeutically effective amount” of the compound of formula (I) when administered as monotherapy may be different from the “WEE1 combination therapeutically effective amount” of the compound of formula (I) when used in combination with TMZ and/or radiotherapy. In the case of cancer, the therapeutically effective amount of the given drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; reduction in corticosteroid dose need; and/or result in a favorable response such as increased Overall Response (OR), Objective Response Rate (ORR), Best Overall Response (BOR), Duration of Response (DOR), Disease Control Rate (DCR), progression-free survival (PFS), overall survival (OS), complete response (CR) rate, partial response (PR) rate, or, in some cases, stable disease (SD) rate, a decrease in progressive disease (PD), an increased time to tumor progression (TTP) or any combination thereof. See the definition herein of “treating”.

As used herein, “RANO criteria” refers to the Response Assessment in Neuro-Oncology recommendations set out in “Updated response assessment criteria for high-grade gliomas: response assessment in neuro-oncology working group” by Wen P Y, et al. 2010, J Clin Oncol 28(11): 1963-72 (“RANO 2010”). In some embodiments, the RANO guideline may evolve in the future and (a) new version(s) may be released and considered.

As used herein, “mRANO criteria” refers to the Modified Response Assessment in Neuro-Oncology recommendations set out in “Modified Criteria for Radiographic Response Assessment in Glioblastoma Clinical Trials” by Ellington B M, et al. 2017, Neurotherapeutics, 14(2):307-20 (“mRANO 2017”). In some embodiments, the mRANO guideline may evolve in the future and (a) new version(s) may be released and considered.

As used herein, “Progression free survival” (PFS) in a clinical trial refers to the time from enrollment, first administration or randomization until disease progression or death from any cause, whichever occurs first. In the field of glioma, PFS is generally measured using the RANO criteria, for example, as per the RANO 2010 or mRANO 2017 criteria, and generally summarized using the Kaplan-Meier method.

As used herein, “Time to Tumor Progression” (TTP) in a clinical trial refers to the time from enrollment, first administration or randomization to disease progression. In the field of glioma, TTP is generally measured using the RANO criteria, for example, as per the RANO 2010 or mRANO 2017 criteria.

As used herein, a “complete response” or “complete remission” or “CR” in a clinical trial indicates that there is no detectable evidence of tumor in response to treatment. This does not always mean the cancer has been cured. Complete response in brain tumors such as glioma is generally measured using the RANO criteria. For example, as per the RANO 2010 or mRANO 2017 criteria, a Complete Response requires all of the following: complete disappearance of all enhancing measurable and non-measurable disease sustained for at least 4 weeks; no new lesions; stable or improved non-enhancing (T2/FLAIR) lesions; and patient must be off corticosteroids or on physiologic replacement doses only, and stable or improved clinically. In the absence of a confirming scan 4 weeks later, this response will be considered only stable disease. Fluid-attenuated inversion recovery (FLAIR) is a magnetic resonance imaging (MRI) sequence that produces strong T2 weighting, suppresses the CSF signal, and minimizes contrast between gray matter and white matter.

As used herein, a “partial response” or “PR” in a clinical trial refers to a decrease in the size or volume of one or more tumors or lesions, or in the extent of cancer in the body, in response to treatment according to the RANO criteria. For example, as per the RANO 2010 or mRANO 2017 criteria, a Partial Response requires all of the following: >=50% decrease, compared with baseline, in the sum of products of perpendicular diameters of all measurable enhancing lesions sustained for at least 4 weeks; no progression of non-measurable disease; no new lesions; stable or improved non-enhancing (T2/FLAIR) lesions on same or lower dose of corticosteroids compared with baseline scan; and patient must be on a corticosteroid dose not greater than the dose at time of baseline scan and is stable or improved clinically. In the absence of a confirming scan 4 weeks later, this response will be considered only stable disease.

As used herein, a “Stable disease” or “SD” in a clinical trial refers to disease without progression or relapse. In stable disease there is neither sufficient tumor shrinkage to qualify for partial response nor sufficient tumor increase to qualify as progressive disease taking as reference the smallest sum diameters while on the study. This may be generally measured using the RANO criteria, for example, as per the RANO 2010 or mRANO 2017 criteria.

As used herein, “Progression”, “Progressive disease” or “PD” in a clinical trial or study refers to the appearance of one or more new lesions or tumors and/or the unequivocal progression of existing target and/or non-target lesions and/or at least a 25% increase in the sum of the products of perpendicular diameters of target lesions, taking as reference the smallest sum on study (Note: the appearance of one or more new lesions is also considered progression). Generally measured using the RANO criteria. For example, as per RANO 2010 criteria, Progression is defined by any of the following: 25% increase in sum of the products of perpendicular diameters of enhancing lesions (compared with baseline if no decrease) on stable or increasing doses of corticosteroids; a significant increase in T2/FLAIR non-enhancing lesions on stable or increasing doses of corticosteroids compared with baseline scan or best response after initiation of therapy, not due to comorbid events; the appearance of any new lesions; clear progression of non-measurable lesions; or definite clinical deterioration not attributable to other causes apart from the tumor, or to decrease in corticosteroid dose. Failure to return for evaluation as a result of death or deteriorating condition should also be considered as progression.

As used herein, “Objective Response Rate” or “ORR” refers to the percentage of subjects in a study, clinical trial or treatment group who have a partial or complete response to the treatment. Generally measured using the RANO criteria, for example, as per the RANO 2010 or mRANO 2017 criteria.

As used herein, “Overall Response” or “Objective Response” or “OR” refers to the response to treatment of a given patient in a study, clinical trial or treatment group at a given assessment point. Generally measured using the RANO criteria, for example, as per the RANO 2010 or mRANO 2017 criteria.

As used herein, “Best Overall Response” or “BOR” refers to the best response recorded for a given patient in a study, clinical trial or treatment group from the baseline assessment (enrollment, start of the treatment or randomization) until disease progression/recurrence (taking as reference for progressive disease the smallest measurements recorded since the treatment started) or death from any cause. Generally measured using the RANO criteria, for example, as per the RANO 2010 or mRANO 2017 criteria.

As used herein, “Disease Control Rate” or “DCR” refers to the percentage of patients in a study, clinical trial or treatment group who have achieved complete response, partial response or stable disease to a therapeutic intervention. Generally measured using the RANO criteria, for example, as per the RANO 2010 or mRANO 2017 criteria.

As used herein, “Duration of response” (DoR) refers to the time from earlier response (PR or better) to disease progression or death from any cause.

Generally measured using the RANO criteria, for example, as per the RANO 2010 or mRANO 2017 criteria.

As used herein, “Overall Survival” (OS) in a clinical trial refers to the time from patient enrollment, first treatment administration or randomization to death from any cause or censored at the date last known alive. Improvement in OS includes a prolongation in life expectancy as compared to naive or untreated individuals or patients. Overall survival refers to the situation wherein a patient remains alive for a defined period of time, such as one year, five years, etc., e.g., from the time of randomization or first treatment.

As used herein, the term “pharmaceutical formulation” or “pharmaceutical composition” refers to a preparation which is in such form as to permit the biological activity of the active ingredient to be effective, and which contains no additional components which are unacceptably toxic to a subject to which the formulation would be administered.

As used herein, the expression “oral dosage form” refers to any form of a pharmaceutical composition that is suitable for oral administration.

As used herein with regard to the schedule of administration for a given drug, the expression “intermittent” or “intermittent schedule” refers to a schedule of administration comprising regular repetition periods, wherein each regular repetition period includes one or more predetermined days of administering the drug and one or more other predetermined days of not administering the drug. The expression “regular repetition period” is to be understood as a time period with a predetermined administration schedule, which is repeated one or more times in the course of the glioma treatment. The regular repetition period may be a 28-day treatment cycle, or a week within a 6 or 7 week-treatment. For example, if the duration of the regular repetition period is of n days, the number of predetermined days of administering the drug may be selected from 1 to n−1 days, the remaining days being the predetermined days of not administering the drug. If the number of predetermined days of administering the drug is 2 or more, these predetermined days may be consecutive or not. In the context of the present application, “n” can be preferably 28 days or 7 days (a week). An intermittent schedule of compound of formula (I) may for instance be administration on days 1 to 5, 8 to 10 and 15 to 17 of a 28-day cycle; on days 1 to 5, 8 to 12 and 15 to 19 of a 28-day cycle; on days 1 to 5, 8 to 12, 15 to 19 and 22-26 of a 28-day cycle; on days 1 to 3 of each week, for 6 or 7 weeks; on days 1 to 5 of each week, for 6 or 7 weeks; or on days 1 to 4 of each week, for 6 or 7 weeks.

As used herein with regard to the schedule of administration for a given drug, the expression “continuous” or “continuous schedule” refers to an administration schedule, wherein the drug is administered on every day of a regular repetition period. A continuous schedule may for instance be daily administration in one or more 28-day cycles; daily administration over a 6 or 7 week-treatment; or daily administration for 42 to 49 days.

### Methods and Uses for the Treatment

In an embodiment of the present invention, the compound of formula (I)

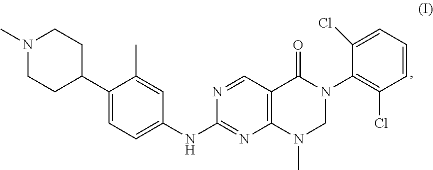 [SMILES: Cc1cc(Nc2ncc3c(n2)N(C)CN(c2c(Cl)cccc2Cl)C3=O)ccc1C1CCN(C)CC1]

or a pharmaceutically acceptable salt thereof, is used for the treatment of glioma in patients in need thereof, in monotherapy.

In another embodiment of the present invention, the compound of formula (I), or a pharmaceutically acceptable salt thereof, is used for the treatment of glioma in patients in need thereof, in combination with TMZ.

In yet another embodiment of the present invention, the compound of formula (I), or a pharmaceutically acceptable salt thereof, is used for the treatment of glioma in patients in need thereof, in combination with radiotherapy.

In yet another embodiment of the present invention, the compound of formula (I), or a pharmaceutically acceptable salt thereof, is used for the treatment of glioma in patients in need thereof, in combination with TMZ and radiotherapy.

In some embodiments of the uses and methods of the present invention, the glioma is treated irrespective of its WHO grade.

In some embodiments of the uses and methods of the present invention, the glioma is grade 1 as per WHO 2021. In other embodiments of the uses and methods of the present invention, the glioma is grade 2 as per WHO 2021. In other embodiments of the uses and methods of the present invention, the glioma is grade 3 as per WHO 2021. In other embodiments of the uses and methods of the present invention, the glioma is grade 4 as per WHO 2021.

In some embodiments of the uses or methods of the present invention, the glioma is treated irrespective of its isocitrate dehydrogenase (IDH) status (e.g. wildtype or mutant).

In some embodiments of the uses or methods of the present invention, the glioma is (IDH)-wildtype. In alternative embodiments of the uses or methods of the present invention, the glioma is (IDH)-mutant.

In some embodiments of the uses or methods of the present invention, the glioma is treated irrespective of its MGMT status.

In some embodiments of the uses or methods of the present invention, the glioma presents a methylated MGMT promoter. In alternative embodiments of the uses or methods of the present invention, the glioma presents an unmethylated MGMT promoter.

In some embodiments of the uses or methods of the present invention, the glioma is treated irrespective of its 1p/19q status.

In some embodiments of the uses or methods of the present invention, the glioma is 1p/19q-codeleted. In alternative embodiments of the uses or methods of the present invention, the glioma is 1p/19q-intact.

In some embodiments of the uses or methods of the present invention, the glioma is treated irrespective of its CDKN2A/B status.

In some embodiments of the uses or methods of the present invention, the glioma carries a homozygous deletion of CDKN2A/B. In alternative embodiments of the uses or methods of the present invention, the glioma retains CDKN2A/B.

In some embodiments of the uses or methods of the present invention, the glioma is treated irrespective of its ATRX status.

In some specific aspects of the present invention, the glioma is ATRX-deficient. In alternative aspects, the glioma retained ATRX. For example, the increased efficacy in treating glioma with the uses or methods of the present invention may be more pronounced in ATRX-deficient glioma, such as when compound of formula (I) is used in monotherapy.

In some specific aspects, the glioma is IDH-wildtype and presents necrosis and/or microvascular proliferation.

In some specific aspects, the glioma is IDH-wildtype in absence of microvascular proliferation or necrosis. For example, the IDH-wild-type glioma may present an EGFR amplification, a combination of gain of chromosome 7 and loss of chromosome 10 (7+/10−), and/or a TERT promoter mutation (pTERTmut).

In some specific aspects of the uses and methods of the present invention, the glioma is treated irrespective of its H3.3 G34 status.

In some specific aspects, the glioma is H3.3 G34 wild type.

In some specific aspects of the uses and methods of the present invention, the glioma is treated irrespective of its H3 K27M status.

In some specific aspects, the glioma is H3 K27M mutant or has a loss of H3K27me3.

The various possible molecular statuses and alterations of a glioma in a patient may be detected or determined independently from each other. The uses or methods of the present invention may be applied to glioma with any molecular status or alteration, particularly to glioma having a single identified molecular alteration, or a combination of 2, 3, 4, 5, 6 or more identified molecular alterations.

For example, the IDH status and the MGMT status may be determined independently from each other in the uses or methods of the present invention and the glioma may be IDH wildtype and MGMT-methylated, IDH wildtype and MGMT-unmethylated, IDH mutant and MGMT-methylated or IDH mutant and MGMT-unmethylated.

In some embodiments of the uses or methods of the present invention, the glioma has recurred or progressed after initial glioma treatment. In some embodiments of the uses or methods of the present invention, the glioma has recurred or progressed after prior glioma treatment.

In alternative embodiments, the patient is naïve of any previous glioma treatment, or the glioma is newly diagnosed.

In some specific aspects of the present invention, the glioma is glioblastoma. For example, the glioma may be a GBM, isocitrate dehydrogenase (IDH)-wildtype, Grade 4 as per WHO 2021.

In other specific aspects of the present invention, the glioma is astrocytoma. For example, the glioma may be astrocytoma IDH-mutant, Grade 2, 3, or 4 as per WHO 2021, preferably Grade 3 as per WHO 2021.

In other specific aspects of the present invention, the glioma is oligodendroglioma. For example, the glioma may be oligodendroglioma, IDH-mutant and 1p/19q-codeleted, Grade 2 or 3 as per WHO 2021.

In some embodiments, the uses or methods of treatment according to the present invention present advantageous properties in increasing efficacy in treating glioma, such as improving OS, PFS, OR, ORR, BOR, DOR, DC and/or DCR as assessed per RANO 2010 or mRANO 2017 criteria, with an acceptable safety profile and no detrimental effect on the patients' quality of life compared with SOC. In the same or other embodiments, the uses or methods of treatment according to the present invention allow to decrease steroid needs in patients suffering from glioma.

### Doses, Schedules and Routes of Administration

As used herein, the term “about” describes a deviation from the indicated value of ±10%. The individual values are to be understood as describing ranges between any of the described values.

### Compound of Formula (I)

In the uses and methods of the present invention, the compound of formula (I) may be administered at doses ranging from about 30 to about 1000 mg of free base, preferably ranging from about 30 to about 720 mg of free base per treatment day. In some embodiments the compound of formula (I) may preferably be administered at doses from about 90 to about 720 mg of free base, or from about 150 to about 720 mg of free base per treatment day.

For example, the compound of formula (I) may be administered at a dose of about 30, about 60, about 75, about 90, about 100, about 120, about 130, about 150, about 200, about 220, about 250, about 260, about 300, about 320, about 350, about 360, about 400, about 420, about 450, about 460, about 500, about 520, about 550, about 600, about 620, about 650, about 700, about 720, about 750, about 800, about 820, about 850, about 900, about 920, about 950 or about 1000 mg of free base per treatment day. Preferably, the compound of formula (I) may be administered at a dose of 30, 60, 75, 90, 100, 120, 130, 150, 200, 220, 250, 260, 300, 320, 350, 360, 400, 420, 450, 460, 500, 520, 550, 600, 620, 650, 700, 720, 750, 800, 820, 850, 900, 920, 950 or 1000 mg of free base per treatment day.

In a more specific embodiment, the compound of formula (I) may be administered at doses of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day. Preferably, the compound of formula (I) may be administered at a dose of 30, 60, 75, 90, 100, 120, 150, 200, 250, 260, 300, 350, 360, 400, 450, 460, 500, 520, 550 or 720 mg of free base per treatment day.

In preferred embodiments, a WEE1 therapeutically effective amount or a WEE1 combination therapeutically effective amount may be administered.

In some embodiments of the present invention, the compound of formula (I) is administered orally.

In some embodiments of the present invention, the compound of formula (I) is administered as a single dose per treatment day (QD), or in two doses per treatment day (BID).

In some embodiments, the compound of formula (I) is administered in an intermittent schedule.

In some embodiments, the compound of formula (I) is administered on Days 1 to 5, 8 to 10 and 15 to 17 of a 28-day cycle. In such embodiments, the compound of formula (I) may be administered for 1, 2, 3, 5, 4, 5, 6 or more cycles. In some aspects, the treatment with compound of formula (I) may continue for 2 years or until progression of disease, unacceptable toxicity, patient's decision to stop, discontinuation as per physician's decision, initiation of subsequent antineoplastic treatment, the end of a clinical study, or death. In some aspects, the treatment with compound of formula (I) may continue for a maximum of 2 years. In some embodiments, the compound of formula (I) may be administered on Days 1 to 5, 8 to 10 and 15 to 17 of a 28-day cycle at the doses per treatment day indicated above.

In some embodiments, the compound of formula (I) is administered on Days 1 to 5, 8 to 12 and 15 to 19 of a 28-day cycle. In such embodiments, the compound of formula (I) may be administered for 1, 2, 3, 5, 4, 5, 6 or more cycles. In some aspects, the treatment with compound of formula (I) may continue for 2 years or until progression of disease, unacceptable toxicity, patient's decision to stop, discontinuation as per physician's decision, initiation of subsequent antineoplastic treatment, the end of a clinical study, or death. In some aspects, the treatment with compound of formula (I) may continue for a maximum of 2 years. In some embodiments, the compound of formula (I) may be administered on Days 1 to 5, 8 to 12 and 15 to 19 of a 28-day cycle at the doses per treatment day indicated above.

In some embodiments, the compound of formula (I) is administered on Days 1 to 5, 8 to 12, 15 to 19 and 22 to 26 of a 28-day cycle. In such embodiments, the compound of formula (I) may be administered for 1, 2, 3, 5, 4, 5, 6 or more cycles. In some aspects, the treatment with compound of formula (I) may continue for 2 years or until progression of disease, unacceptable toxicity, patient's decision to stop, discontinuation as per physician's decision, initiation of subsequent antineoplastic treatment, the end of a clinical study, or death. In some aspects, the treatment with compound of formula (I) may continue for a maximum of 2 years. In some embodiments, the compound of formula (I) may be administered on Days 1 to 5, 8 to 12, 15 to 19 and 22 to 26 of a 28-day cycle at the doses per treatment day indicated above.

In some embodiments, the compound of formula (I) is administered on Days 1 to 3 of each week, for several weeks, in particular for 6 or 7 weeks. In other embodiments, the compound of formula (I) may be administered on Days 1 to 3 of each week for 42 days to 49 days, such as for 42, 43, 44, 45, 46, 47, 48 or 49 days. In some embodiments, the compound of formula (I) may be administered for 6 weeks, for 7 weeks or for 42 to 49 days, at the doses per treatment day indicated above.

In some embodiments, the compound of formula (I) is administered on Days 1 to 5 of each week, for several weeks, in particular for 6 or 7 weeks. In other embodiments, the compound of formula (I) may be administered on Days 1 to 5 of each week for 42 days to 49 days, such as for 42, 43, 44, 45, 46, 47, 48 or 49 days. In some embodiments, the compound of formula (I) may be administered for 6 weeks, for 7 weeks or for 42 to 49 days, at the doses per treatment day indicated above.

In some embodiments, the compound of formula (I) is administered, on Days 1 to 4 of each week, for several weeks, in particular for 6 or 7 weeks. In other embodiments, the compound of formula (I) may be administered on Days 1 to 4 of each week for 42 days to 49 days, such as for 42, 43, 44, 45, 46, 47, 48 or 49 days. In some embodiments, the compound of formula (I) may be administered for 6 weeks, for 7 weeks or for 42 to 49 days, at the doses per treatment day indicated above.

In some embodiments, one or more holiday periods are included in a 6-week schedule for compound of formula (I), such as one or more holiday periods of one week each. A holiday period is to be understood as a period without administration of compound of formula (I). Said holiday periods do not affect administration of TMZ and/or radiotherapy, which are to be continued according to schedules and with the doses specified hereinabove or hereinbelow. The 6-week schedule for compound of formula (I) may be any 6-week schedule described for the compound of formula (I) hereinabove or hereinbelow. For example, the compound of formula (I) may be administered on Days 1 to 3 of weeks 1, 2, 4 and 5, wherein weeks 3 and 6 are holiday periods (with no administration of compound of formula (I)). Or the compound of formula (I) may be administered on Days 1 to 3 of weeks 1, 2, 3, 5 and 6, wherein week 4 is a holiday period (with no administration of compound of formula (I)). In such embodiments including one or more holiday periods, the compound of formula (I) may be administered at the doses per treatment day indicated above.

In other embodiments of the present invention, the compound of formula (I) is administered in a continuous schedule. For example, the compound of formula (I) is administered daily, for several weeks, in particular for 6 or 7 weeks. In some embodiments, the compound of formula (I) may be administered daily for 42 days to 49 days, such as daily for 42, 43, 44, 45, 46, 47, 48 or 49 days. In some embodiments, the compound of formula (I) may be administered daily, for 6 weeks, daily for 7 weeks or daily for 42 to 49 days, at the doses per treatment day indicated above. It may be preferred that the compound of formula (I) (optionally in combination with TMZ in the schedules defined herein) is administered on Days 1 to 5, 8 to 10 and 15 to 17 of a 28-day cycle, or on Days 1 to 5, 8 to 12 and 15 to 19 of a 28-day cycle, or on Days 1 to 5, 8 to 12, 15 to 19 and 22 to 26 of a 28-day cycle, if the glioma to be treated has recurred after initial or prior glioma treatment (second (or higher) line treatment). It may be preferred that the compound of formula (I) (optionally in combination with TMZ and/or radiotherapy in the schedules defined herein) is administered daily, for 6 weeks, daily for 7 weeks or 42 to 49 days, if the glioma to be treated has been newly diagnosed (first line treatment). It may be more preferred that the compound of formula (I) (optionally in combination with TMZ and/or radiotherapy in the schedules defined herein) is administered on Days 1 to 3 of each week for 6 or 7 weeks, or on Days 1 to 5 of each week for 6 or 7 weeks, or on Days 1 to 4 of each week for 6 or 7 weeks, if the glioma to be treated has been newly diagnosed (first line treatment).

In some embodiments of the present invention, the compound of formula (I) is administered at approximately the same time on each treatment day, e.g. at the same time ±about 60 min, preferably ±60 min on each treatment day, for example in a given cycle. In more specific embodiments, the compound of formula (I) is administered in the morning, for example between 5 a.m and noon.

In some aspects of the uses or methods according to the present invention, the compound of formula (I) is administered after fasting, preferably after a fasting period of 4 hours. In the same or other aspects, the compound of formula (I) administration may be followed by fasting, preferably followed by 2 hours of fasting.

In alternative aspects of the uses and methods according to the present invention, the compound of formula (I) is administered in a fed state.

In yet other alternative aspects of the uses or methods according to the present invention, the compound of formula (I) is administered irrespective of food status of the patient.

### Temozolomide (TMZ)

In some embodiments of the methods of the present invention, when TMZ is administered in combination with the compound of formula (I) or in combination with the compound of formula (I) and radiotherapy, TMZ may be administered at a dose ranging from about 50 to about 300 mg/m²/day, preferably about 50 to about 250 mg/m²/day, more preferably about 50 to about 200 mg/m²/day.

In preferred embodiments, a TMZ therapeutically effective amount or a TMZ combination therapeutically effective amount may be administered.

In specific embodiments, TMZ may be administered at a dose of about 75 mg/m²/day, about 150 mg/m²/day, or about 200 mg/m²/day. Preferably, TMZ may be administered at a dose of 75 mg/m²/day, 150 mg/m²/day, or 200 mg/m²/day.

In some embodiments, TMZ may be administered on Days 1 to 5 of a 28-day cycle. In other embodiments, TMZ may be administered on Days 1 to 5, 8 to 10 and 15 to 17 of a 28-day cycle. The 28-day cycle may be the same as the one of compound of formula (I).

TMZ may be administered over 1, 2, 3, 4, 5, 6 or more cycles. In some aspects, treatment with TMZ may continue for 2 years. In some aspects, treatment with TMZ may continue for a maximum of 2 years.

In yet other embodiments, TMZ may be administered daily, for 6 weeks, daily for 7 weeks, or daily for 42 days to 49 days.

In some specific preferred embodiments, TMZ may be administered at a dose ranging from 50 to 300 mg/m²/day, preferably 50 to 250 mg/m²/day, further preferably 50 to 200 mg/m²/day on days 1 to 5 of a 28-day cycle. Preferably, TMZ may be administered at a dose of 150 mg/m²/day or 200 mg/m²/day on days 1 to 5 of a 28-day cycle. In these embodiments, TMZ may be administered over 1, 2, 3, 4, 5, 6 or more cycles. In some aspects, treatment with TMZ may continue for 2 years. In some aspects, treatment with TMZ may continue for a maximum of 2 years. This TMZ schedule may be preferred, if the glioma to be treated has recurred after initial or prior glioma treatment, wherein the compound of formula (I) is administered on the administration days defined above, in particular on Days 1 to 5 of a 28-day cycle.

In other specific preferred embodiments, TMZ may be administered at a dose of 50 to 300 mg/m²/day, preferably 50 to 250 mg/m²/day, further preferably 50 to 200 mg/m²/day, daily for 6 weeks, daily for 7 weeks, or daily for 42 days to 49 days. Preferably, TMZ may be administered at a dose of 75 mg/m²/day daily, for 6 weeks, daily for 7 weeks, or daily for 42 days to 49 days. This TMZ schedule may be preferred, if the glioma to be treated has been newly diagnosed, wherein the compound of formula (I) is administered on the administration days defined above, in particular daily for 6 weeks, daily for 7 weeks or daily for 42 to 49 days. For the treatment of newly diagnosed glioma, radiotherapy may be administered in addition, as defined further below.

In other embodiments, TMZ may be administered at a dose ranging from 50 to 300 mg/m²/day, preferably 50 to 250 mg/m²/day, further preferably 50 to 200 mg/m²/day on Days 1 to 5, 8 to 10 and 15 to 17 of a 28-day cycle. Preferably, TMZ may be administered at a dose of 75 mg/m²/day, 150 mg/m²/day, or 200 mg/m²/day on Days 1 to 5, 8 to 10 and 15 to 17 of a 28-day cycle. This TMZ schedule may be used, if the glioma to be treated has recurred after initial or prior glioma treatment, wherein the compound of formula (I) is administered on the same administration days, i.e. both the compound of formula (I) and TMZ are administered on Days 1 to 5, 8 to 10 and 15 to 17 of a 28-day cycle.

TMZ may be administered on the same days as the compound of formula (I), or in a different schedule, as defined below.

In some embodiments, TMZ is administered in a different schedule to the compound of formula (I).

In one example, if the compound of formula (I) is administered on Days 1 to 5, 8 to 10 and 15 to 17 of a 28-day cycle, TMZ may be administered on Days 1 to 5 of the 28-day cycle. In another example, if the compound of formula (I) is administered on Days 1 to 5, 8 to 12 and 15 to 19 of a 28-day cycle, TMZ may be administered on Days 1 to 5 of the 28-day cycle. In yet another example, if the compound of formula (I) is administered on Days 1 to 5, 8 to 12, 15 to 19 and 22 to 26 of a 28-day cycle, TMZ may be administered on Days 1 to 5 of the 28-day cycle. The dosages of Compound of formula (I) and of TMZ per respective administration day may be as defined above. This schedule may be preferred, if the glioma to be treated has recurred after initial or prior glioma treatment.

In yet other examples: if the compound of formula (I) is administered on Days 1 to 3 of each week for 6 or 7 weeks, or 42 days to 49 days, TMZ may be administered daily for said 6 weeks, 7 weeks, or 42 days to 49 days; if the compound of formula (I) is administered on Days 1 to 5 of each week for 6 or 7 weeks, or 42 days to 49 days, TMZ may be administered daily for said 6 weeks, 7 weeks, or 42 days to 49 days; if the compound of formula (I) is administered or on Days 1 to 4 of each week for 6 or 7 weeks, or 42 days to 49 days, TMZ may be administered daily for said 6 weeks, 7 weeks, or 42 days to 49 days. The dosages of Compound of formula (I) and of TMZ on the administration days may be as defined above. These schedules having a duration of 6 or 7 weeks or 42 to 49 days may be preferred if the glioma to be treated has been newly diagnosed.

In other embodiments, TMZ is administered in the same schedule as the compound of formula (I). In one example, if the compound of formula (I) is administered daily for 6 weeks, daily for 7 weeks, or daily for 42 days to 49 days, TMZ may also be administered daily for said 6 weeks, 7 weeks, or 42 days to 49 days. The daily dosages of Compound of formula (I) and of TMZ on the administration days may be as defined above. This schedule may be preferred, if the glioma to be treated has been newly diagnosed.

In another example, if the compound of formula (I) is administered on Days 1 to 5, 8 to 10 and 15 to 17 of a 28-day cycle, TMZ may also be administered on Days 1 to 5, 8 to 10 and 15 to 17 of the 28-day cycle. The daily dosages of Compound of formula (I) and of TMZ on the administration days may be as defined above. This schedule may be preferred, if the glioma to be treated has recurred after initial or prior glioma treatment.

Specific TMZ schedules that can be combined with compound of formula (I) in the uses and methods according to the present invention include:
a) 75 mg TMZ/m²/day daily for 6 weeks, daily for 7 weeks or daily for 42-49 days,
b) 150 mg TMZ/m²/day on days 1-5 of each 28-day cycle,
c) 200 mg TMZ/m²/day on days 1-5 of each 28-day cycle,
d) 50 mg TMZ/m²/day, daily, until progression,
e) 1 week on TMZ (e.g. 120 mg/m² per day, daily for 7 days)/1 week off (7 days without TMZ administration), in a 28-day cycle,
f) 3 weeks on TMZ (e.g. 80 mg/m² per day, daily for 21 days)/1 week off (7 days without TMZ administration), in a 28-day cycle, or
g) 75 mg TMZ/m²/day on days 1 to 5 of each week, for 6 or 7 weeks, or for 42 to 49 days.

In some embodiments, it may be desired to express the amount of TMZ to be administered in mg per treatment day. In such embodiments, the daily dose may be calculated based on the indicated mg/m²/day and the body surface area (BSA) of the patient. The BSA may be determined by the skilled person by methods known to him, such as by the Du Bois method (see e.g. Dubois D, Dubois E F. A formula to estimate the approximate surface area if height and weight be known. Arch Intern Med. 1916; 17:863-871) or the Mosteller formula (see e.g. Mosteller RD. Simplified calculation of body-surface area. N Engl J Med 1987; 317:1098).

Table 1 below provides an overview on how the 75 mg/m²/day, 150 mg/m²/day, or 200 mg/m²/day dosages may be converted into mg per treatment day, depending on the BSA. For instance, for a patient having a BSA of 2.0, a dosage of 75 mg/m²/day would mean that 150 mg total of TMZ may be administered.

TABLE 1
Daily Dose Calculations of TMZ by Body Surface Area (BSA)

| | 75 mg/m2 | 150 mg/m2 | 200 mg/m2 |
|---|---|---|---|
| Total BSA (m2) | (mg daily) | (mg daily) | mg daily) |
| 1.0 | 75 | 150 | 200 |
| 1.1 | 82.5 | 165 | 220 |
| 1.2 | 90 | 180 | 240 |
| 1.3 | 97.5 | 195 | 260 |
| 1.4 | 105 | 210 | 280 |
| 1.5 | 112.5 | 225 | 300 |
| 1.6 | 120 | 240 | 320 |
| 1.7 | 127.5 | 255 | 340 |
| 1.8 | 135 | 270 | 360 |
| 1.9 | 142.5 | 285 | 380 |
| 2.0 | 150 | 300 | 400 |
| 2.1 | 157.5 | 315 | 420 |
| 2.2 | 165 | 330 | 440 |
| 2.3 | 172.5 | 345 | 460 |
| 2.4 | 180 | 360 | 480 |
| 2.5 | 187.5 | 375 | 500 |

In some embodiments of the present invention, TMZ is administered orally. In alternative embodiments of the present invention, TMZ is administered intravenously by infusion. The dose for TMZ administered as an intravenous infusion (e.g. over 90 minutes) is generally the same as the dose for the oral formulation.

Of course, in some embodiments, TMZ may be used in the form of a pharmaceutically acceptable salt. In such embodiments, TMZ indications of dosages, amounts or concentrations herein, which are indications for the free base, may be suitably adapted in view of the molecular weight of the respective salt.

### Radiotherapy

In some embodiments of the methods of the present invention, radiotherapy is used in combination with compound of formula (I) only, or in combination with compound of formula (I) and TMZ. The use of radiotherapy may generally be preferred, if the glioma to be treated has been newly diagnosed (first line treatment).

The radiotherapy used may be any suitable type of radiation therapy, such as 3D conformal radiation therapy (3DCRT), intensity modulated radiation therapy (IMRT), focal radiotherapy or fractionated radiotherapy.

In some embodiments, radiotherapy is fractionated radiotherapy.

In preferred embodiments, radiotherapy is administered over 5 consecutive days per week until a total radiation dose has been administered. In another preferred embodiment, radiotherapy is administered in equal fractions over a period of 6 weeks, 7 weeks or 42 to 49 days. It may be understood that radiotherapy does not have to be administered daily.

In one example, if the compound of formula (I) is administered daily, for 6 weeks, daily for 7 weeks, or daily for 42 days to 49 days, radiotherapy may be administered in equal fractions over the period of 6 weeks, 7 weeks or 42 to 49 days. In a further example, if the compound of formula (I) and TMZ are administered daily for 6 weeks, daily for 7 weeks, or daily for 42 days to 49 days, radiotherapy may be administered in equal fractions over the period of 6 weeks, 7 weeks or 42 to 49 days. The dosages of Compound of formula (I) and/or of TMZ per respective administration day may be as defined above.

Or, if the compound of formula (I) is administered on Days 1 to 3 of each week for 6 or 7 weeks or for 42 days to 49 days, radiotherapy may be administered in equal fractions over the period of 6 weeks, 7 weeks, or 42 to 49 days. In a further example, if the compound of formula (I) is administered on Days 1 to 3 of each week for 6 or 7 weeks or for 42 days to 49 days, and TMZ is administered daily for 6 weeks, daily for 7 weeks, or daily for 42 days to 49 days, radiotherapy may be administered in equal fractions over the period of 6 weeks, 7 weeks or 42 to 49 days. The dosages of Compound of formula (I) and/or of TMZ per respective administration day may be as defined above.

Or, if the compound of formula (I) is administered on Days 1 to 5 of each week for 6 or 7 weeks or for 42 days to 49 days, radiotherapy may be administered in equal fractions over the period of 6 weeks, 7 weeks, or 42 to 49 days. In a further example, if the compound of formula (I) is administered on Days 1 to 5 of each week for 6 or 7 weeks or for 42 days to 49 days, and TMZ is administered daily for 6 weeks, daily for 7 weeks, or daily for 42 days to 49 days, radiotherapy may be administered in equal fractions over the period of 6 weeks, 7 weeks or 42 to 49 days. The dosages of Compound of formula (I) and/or of TMZ per respective administration day may be as defined above.

Or, if the compound of formula (I) is administered on Days 1 to 4 of each week for 6 or 7 weeks or for 42 days to 49 days, radiotherapy may be administered in equal fractions over the period of 6 weeks, 7 weeks, or 42 to 49 days. In a further example, if the compound of formula (I) is administered on Days 1 to 4 of each week for 6 or 7 weeks or for 42 days to 49 days, and TMZ is administered daily for 6 weeks, daily for 7 weeks, or daily for 42 days to 49 days, radiotherapy may be administered in equal fractions over the period of 6 weeks, 7 weeks or 42 to 49 days. The dosages of Compound of formula (I) and/or of TMZ per respective administration day may be as defined above.

In the same or other embodiments, radiotherapy is administered in a regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks or 42 to 49 days.

In particular preferred embodiments, radiotherapy is administered in a regimen of 2 Gy per fraction daily, 5 consecutive days per week for a total of 60 Gy, and/or at a total dose of 60 Gy over 30 fractions, over a period of 6 weeks, 7 weeks or 42 to 49 days.

In some aspects, the administration schedules described herein, for example when involving radiotherapy, may be understood to cover administration schedules which have been interrupted for one or several days (consecutive or not). A cause for interruption may for instance be a broken radiotherapy machine. On such interruption days, RT, compound of formula (I) and TMZ, if scheduled to be administered on said days, are interrupted. The days on which the administration has been interrupted may be added at the end of the treatment period. For example, the administration may be interrupted on one day of a 6-week (42-day) treatment period and the missed administration day may then be added during the 7^{th} week (after day 42), preferably on the first day of the 7^{th} week (on day 43). In case of several missed administration days, the overall treatment period may be extended by the number of missed administration days up to a maximum of 7 weeks (49 days). The specific handling of missed administration days is decided upon by the treating physician or the physician team around a glioma patient according to their expertise and the patient's condition.

### Combination Treatments Involving Compound of Formula (I)

In some embodiments of the present invention where the compound of formula (I) is used in combination with TMZ and/or radiotherapy, if TMZ and/or radiotherapy are administered on said WEE1 inhibitor treatment day, administration of compound of formula (I) precedes administration of TMZ and/or radiotherapy, on a WEE1 inhibitor treatment day.

In some embodiments, on a WEE1 inhibitor treatment day, if TMZ is administered on said WEE1 inhibitor treatment day, TMZ administration is initiated about 30 minutes to 4 hours, preferably about 30 minutes to 1 hour, after administration of compound of formula (I). In alternative embodiments, on a WEE1 inhibitor treatment day, if TMZ is administered on said WEE1 inhibitor treatment day, TMZ is administered at bedtime, such as between 9 pm and midnight.

In some embodiments of the present invention where the compound of formula (I) is used in combination with both TMZ and radiotherapy, on a WEE1 inhibitor treatment day, if TMZ and/or radiotherapy are administered on said WEE1 inhibitor treatment day, TMZ administration is initiated about 30 minutes to 4 hours, preferably about 30 minutes to 1 hour, after administration of compound of formula (I), and radiotherapy is initiated about 1 hour after the end of TMZ administration.

In some embodiments of the present invention where the compound of formula (I) is used in combination with radiotherapy only, if radiotherapy is administered on said WEE1 inhibitor treatment day, radiotherapy is initiated about 30 minutes to 4 hours, preferably about 30 minutes to 1 hour, after compound of formula (I) administration.

Glioma patients may be in need of steroid support. In some embodiments of the present invention, patients in need thereof are administered steroids in the context of their glioma treatment, such as in the context of a first or prior glioma treatment. In some embodiments of the present invention, glioma patients in need thereof are administered steroids in addition to the combination treatment of compound of formula (I) with TMZ, compound of formula (I) with TMZ and RT, or compound of formula (I) with RT. In particular embodiments, the steroids are corticosteroids, such as, but not limited to, dexamethasone or prednisone. In particular embodiments, the maximum daily dose for dexamethasone is 8 mg, preferably 4 mg, or the maximum daily dose for prednisone is 40 mg, preferably 25 mg.

Glioma patients may also be in need of anticonvulsant support, for example in case of seizures. In some embodiments of the present invention, glioma patients in need thereof are administered anticonvulsants in addition to the combination treatment of compound of formula (I) with TMZ, compound of formula (I) with TMZ and RT, or compound of formula (I) with RT. In particular embodiments, the anticonvulsants are devoid of enzyme inhibiting or inducing properties, such as, but not limited to, lacosamide, gabapentin, or pregabalin.

In some embodiments of the present invention, such as when the patients receive concomitant TMZ and RT treatments, Pneumocystis jirovecii pneumonia (PCP) prophylaxis is administered to the patients in addition to the combination treatment of compound of formula (I) with TMZ and RT.

In some aspects of the uses or methods according to the present invention, such as (but not limited to) where the glioma is newly diagnosed, the combined treatment schedule or regimen may be defined as in the following alternative or complementary items:
a) Compound of formula (I) administered daily, for 6 weeks, daily for 7 weeks or daily for 42 days to 49 days, in combination with TMZ administered daily, for 6 weeks, daily for 7 weeks or daily for 42 days to 49 days, with concomitant radiotherapy for 6 weeks, 7 weeks or for 42 days to 49 days;
b) Compound of formula (I) administered daily, for 6 weeks, daily for 7 weeks, or daily for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
c) Compound of formula (I) administered daily, for 6 weeks, daily for 7 weeks, or daily for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   TMZ is administered at a dose of 50-300 mg/m²/day, preferably 50-250 mg/m²/day, further preferably 50-200 mg/m²/day, most preferably 75 mg/m²/day;
d) Compound of formula (I) administered daily, for 6 weeks, daily for 7 weeks, or daily for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 50-300 mg/m²/day, preferably 50-250 mg/m²/day, further preferably 50-200 mg/m²/day, most preferably 75 mg/m²/day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
e) Compound of formula (I) administered daily, for 6 weeks, daily for 7 weeks, or daily for 42 days to 49 days, in combination with TMZ administered daily for 6 weeks, daily for 7 weeks, or daily for 42 days to 49 days, with concomitant radiotherapy for 6 weeks, 7 weeks, or for 42 days to 49 days, wherein
   compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 75 mg/m²/day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks or 42 to 49 days;
f) Compound of formula (I) administered daily, for 6 weeks, daily for 7 weeks, or daily for 42 days to 49 days, in combination with radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
g) Compound of formula (I) administered daily, for 6 weeks, daily for 7 weeks, or daily for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
h) Compound of formula (I) administered daily, for 6 weeks, daily for 7 weeks, or daily for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks, or 42 days to 49 days.

In some aspects, an intermittent schedule may be used in cases where unacceptable toxicity is observed upon continuous administration (i.e. daily).

In some aspects of the uses or methods according to the present invention, such as (but not limited to) where the glioma is newly diagnosed, the combined treatment schedule or regimen may be defined as in the following alternative or complementary items:
aa) Compound of formula (I) administered on Days 1 to 3 of each week for 6 or 7 weeks or for 42 days to 49 days, in combination with TMZ administered daily, for 6 weeks, daily for 7 weeks or daily for 42 days to 49 days, with concomitant radiotherapy for 6 weeks, 7 weeks or for 42 days to 49 days;
bb) Compound of formula (I) administered on Days 1 to 3 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
cc) Compound of formula (I) administered on Days 1 to 3 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   TMZ is administered at a dose of 50-300 mg/m²/day, preferably 50-250 mg/m²/day, further preferably 50-200 mg/m²/day, most preferably 75 mg/m²/day;
dd) Compound of formula (I) administered on Days 1 to 3 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 50-300 mg/m²/day, preferably 50-250 mg/m²/day, further preferably 50-200 mg/m²/day, most preferably 75 mg/m²/day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
ee) Compound of formula (I) administered on Days 1 to 3 of each week for 6 or 7 weeks or for 42 days to 49 days, in combination with TMZ administered daily for 6 weeks, daily for 7 weeks, or daily for 42 days to 49 days, with concomitant radiotherapy for 6 weeks, 7 weeks, or for 42 days to 49 days, wherein
   compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 75 mg/m²/day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks or 42 to 49 days;
ff) Compound of formula (I) administered on Days 1 to 3 of each week for 6 or 7 weeks or for 42 days to 49 days, in combination with radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
gg) Compound of formula (I) administered on Days 1 to 3 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
hh) Compound of formula (I) administered on Days 1 to 3 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks, or 42 days to 49 days.

In some aspects of the uses or methods according to the present invention, such as (but not limited to) where the glioma is newly diagnosed, the combined treatment schedule or regimen may be defined as in the following alternative or complementary items:
aaa) Compound of formula (I) administered on Days 1 to 5 of each week for 6 or 7 weeks or for 42 days to 49 days, in combination with TMZ administered daily, for 6 weeks, daily for 7 weeks or daily for 42 days to 49 days, with concomitant radiotherapy for 6 weeks, 7 weeks or for 42 days to 49 days;
bbb) Compound of formula (I) administered on Days 1 to 5 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
ccc) Compound of formula (I) administered on Days 1 to 5 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   TMZ is administered at a dose of 50-300 mg/m²/day, preferably 50-250 mg/m²/day, further preferably 50-200 mg/m²/day, most preferably 75 mg/m²/day;
ddd) Compound of formula (I) administered on Days 1 to 5 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 50-300 mg/m²/day, preferably 50-250 mg/m²/day, further preferably 50-200 mg/m²/day, most preferably 75 mg/m²/day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
eee) Compound of formula (I) administered on Days 1 to 5 of each week for 6 or 7 weeks or for 42 days to 49 days, in combination with TMZ administered daily for 6 weeks, daily for 7 weeks, or daily for 42 days to 49 days, with concomitant radiotherapy for 6 weeks, 7 weeks, or for 42 days to 49 days, wherein
   compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 75 mg/m²/day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks or 42 to 49 days;
fff) Compound of formula (I) administered on Days 1 to 5 of each week for 6 or 7 weeks or for 42 days to 49 days, in combination with radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
ggg) Compound of formula (I) administered on Days 1 to 5 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
hhh) Compound of formula (I) administered on Days 1 to 5 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks, or 42 days to 49 days.

In some aspects of the uses or methods according to the present invention, such as (but not limited to) where the glioma is newly diagnosed, the combined treatment schedule or regimen may be defined as in the following alternative or complementary items:
aaaa) Compound of formula (I) administered on Days 1 to 4 of each week for 6 or 7 weeks or for 42 days to 49 days, in combination with TMZ administered daily, for 6 weeks, daily for 7 weeks or daily for 42 days to 49 days, with concomitant radiotherapy for 6 weeks, 7 weeks or for 42 days to 49 days;
bbbb) Compound of formula (I) administered on Days 1 to 4 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
cccc) Compound of formula (I) administered on Days 1 to 4 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   TMZ is administered at a dose of 50-300 mg/m²/day, preferably 50-250 mg/m²/day, further preferably 50-200 mg/m²/day, most preferably 75 mg/m²/day;
dddd) Compound of formula (I) administered on Days 1 to 4 of each week for 6 or 7 weeks or for 42 days to 49 days, in combination with TMZ administered daily, for 6 weeks, daily for 7 weeks, or daily for 42 days to 49 days, with concomitant radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 50-300 mg/m²/day, preferably 50-250 mg/m²/day, further preferably 50-200 mg/m²/day, most preferably 75 mg/m²/day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
eeee) Compound of formula (I) administered on Days 1 to 4 of each week for 6 or 7 weeks or for 42 days to 49 days, in combination with TMZ administered daily for 6 weeks, daily for 7 weeks, or daily for 42 days to 49 days, with concomitant radiotherapy for 6 weeks, 7 weeks, or for 42 days to 49 days, wherein
   compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day,
   TMZ is administered at a dose of 75 mg/m²/day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks or 42 to 49 days;
ffff) Compound of formula (I) administered on Days 1 to 4 of each week for 6 or 7 weeks or for 42 days to 49 days, in combination with radiotherapy for 6 weeks, 7 weeks, or for 42 days to 49 days, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
gggg) Compound of formula (I) administered on Days 1 to 4 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 1 Gy to 100 Gy, preferably 2 Gy to 80 Gy, further preferably 10 Gy to 60 Gy, and even more preferably 60 Gy divided over 5 consecutive days per week until a total radiation dose has been administered, or in equal fractions over a period of 6 weeks, 7 weeks, or 42 days to 49 days, most preferably in a fractionated regimen of 2 Gy per fraction daily, 5 consecutive days per week, for a total dose of 60 Gy over 30 fractions, or at a total dose of 60 Gy over 30 fractions, for example over a period of 6 weeks, 7 weeks, or 42 days to 49 days;
hhhh) Compound of formula (I) administered on Days 1 to 4 of each week for 6 or 7 weeks or for 42 days to 49 days, in combination with radiotherapy for 6 weeks, 7 weeks, or for 42 days to 49 days, wherein
   the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and
   radiotherapy is administered in a fractionated regimen of 2 Gy per fraction daily, 5 consecutive days per week, for a total dose of 60 Gy over 30 fractions, or at a total dose of 60 Gy over 30 fractions, for example over a period of 6, 7 weeks, or 42 days to 49 days.

In alternative embodiments of any one of the above-mentioned combination treatments involving compound of formula (I), such as items a) to h), aa) to hh), aaa) to hhh), and aaaa) to hhhh), one or more holiday periods are included in a 6-week schedule for compound of formula (I), such as one or more holiday periods of one week each. A holiday period is to be understood as a period without administration of compound of formula (I). Said holiday periods do not affect administration of TMZ and/or radiotherapy, which are to be continued according to schedule and with the doses specified hereinabove or hereinbelow. For example, in alternatives to items aa) to hh), the compound of formula (I) may be administered on Days 1 to 3 of weeks 1, 2, 4 and 5, wherein weeks 3 and 6 are holiday periods (with no administration of compound of formula (I)). Or the compound of formula (I) may be administered on Days 1 to 3 of weeks 1, 2, 3, 5 and 6, wherein week 4 is a holiday period (with no administration of compound of formula (I)).

In some embodiments of any of the above-mentioned items a) to h), aa) to hh), aaa) to hhh), and aaaa) to hhhh), or of the alternative embodiments including the holiday periods, compound of formula (I) may preferably be administered at a dose ranging from about 90 to about 720 mg of free base.

In other embodiments of any of the above-mentioned items a) to h), aa) to hh), aaa) to hhh), and aaaa) to hhhh), or of the alternative embodiments including the holiday periods, compound of formula (I) may preferably be administered at a dose ranging from about 30 to about 200 mg of free base, or 60 to 200 mg of free base.

In other alternative embodiments of any one of the above-mentioned combination treatments involving compound of formula (I), such as items a) to h), aa) to hh), aaa) to hhh), and aaaa) to hhhh), TMZ may not be administered daily, but on days 1 to 5 of each week, for 6 or 7 weeks, or on days 1 to 5 of each week for 42 to 49 days, preferably at 75 mg TMZ/m²/day on days 1 to 5 of each week, for 6 or 7 weeks, or on days 1 to 5 of each week for 42 to 49 days.

In any of the above-mentioned items a) to h), aa) to hh), aaa) to hhh), and aaaa) to hhhh), or in the alternative embodiments including the holiday periods, the 6-week, 7-week or 42-49 day combination treatment regimen involving compound of formula (I) may be followed by a maintenance treatment with TMZ, optionally with addition of alternating, electric, tumor-treating fields (TTFields). In more specific aspects, the 6-week, 7-week or 42-49 day combination treatment regimen involving compound of formula (I) may be followed by one or more 28-day cycles of a maintenance treatment with TMZ, wherein TMZ is administered on days 1 to 5 of each 28-day cycle. For example, the maintenance treatment with TMZ comprises 6 cycles, e.g. of 28 days. Optionally, alternating, electric, tumor-treating fields (TTFields) may be added to the TMZ maintenance treatment. Optionally, a 4-to-6-week rest period is observed before initiating the TMZ maintenance treatment.

In specific embodiments of any of the above-mentioned combination treatments involving compound of formula (I), such as items a) to h), aa) to hh), aaa) to hhh), and aaaa) to hhhh), optionally followed by a TMZ maintenance treatment, the glioma is newly diagnosed. In more specific embodiments, the newly diagnosed glioma is Grade 4 as per WHO 2021. In the same or other specific embodiments, the newly diagnosed glioma is IDH-wildtype.

In some aspects of the uses or methods according to the present invention, such as (but not limited to) where the glioma has recurred after initial or prior glioma treatment, the combined treatment may be defined as in the following alternative or complementary items:
k) Compound of formula (I) administered on days 1-5, 8-10, and 15-17 of a 28-day cycle, in combination with TMZ;
l) Compound of formula (I) administered on days 1-5, 8-10, and 15-17 of a 28-day cycle, in combination with TMZ, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day;
m) Compound of formula (I) administered on days 1-5, 8-10, and 15-17 of a 28-day cycle, in combination with TMZ, wherein compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and wherein TMZ is administered
   at a dose of 50-300 mg/m²/day, preferably 50-250 mg/m²/day, further preferably 50-200 mg/m²/day, even further preferably 150 to 200 mg/m²/day on days 1-5 of each 28-day cycle, or
   at a dose of 50 mg/m²/day daily, until progression, or
   on days 1-7 of each 28-day cycle, for example at 120 mg/m²/day, or
   on days 1-21 of each 28-day cycle, for example at 80 mg/m²/day;
n) Compound of formula (I) administered on days 1-5, 8-10, and 15-17 of a 28-day cycle, in combination with TMZ, wherein the compound of formula (I) is administered at a dose ranging from about 30 to about 1000 mg, preferably about 30 to about 720 mg of free base per treatment day, most preferably at a dose of about 30, about 60, about 75, about 90, about 100, about 120, about 150, about 200, about 250, about 260, about 300, about 350, about 360, about 400, about 450, about 460, about 500, about 520, about 550 or about 720 mg of free base per treatment day, and wherein TMZ is administered
   at a dose ranging from 150 to 200 mg/m²/day on days 1-5 of each 28-day cycle, or
   at a dose of 50 mg/m²/day daily, until progression, or
   on days 1-7 of each 28-day cycle, for example at 120 mg/m²/day, or
   on days 1-21 of each 28-day cycle, for example at 80 mg/m²/day.

In alternative embodiments derived from any one of the above-mentioned items k) to n), compound of formula (I) may be additionally administered on days 11-12 and 18-19 of the 28-day cycle, i.e. the compound of formula (I) may be administered on days 1-5, 8-12, and 15-19 of a 28-day cycle (additional WEE1 inhibitor treatment days).

In further alternative embodiments derived from any one of the above-mentioned items k) to n), compound of formula (I) may be additionally administered on days 11-12, 18-19 and 22-26 of the 28-day cycle, i.e. the compound of formula (I) may be administered on days 1-5, 8-12, 15-19 and 22-26 of a 28-day cycle (additional WEE1 inhibitor treatment days).

In some embodiments of any of the above-mentioned items k) to n), or of any one of the embodiments with the additional WEE1 inhibitor treatment days, compound of formula (I) may preferably be administered at a dose ranging from about 150 to about 720 mg of free base, or from about 200 to about 720 mg of free base.

In any of the above-mentioned items k) to n), or in any one of the embodiments with the additional WEE1 inhibitor treatment days, compound of formula (I) may be administered for 1, 2, 3, 4, 5, 6 or more 28-day cycles. For example, compound of formula (I) may be administered for up to 2 years.

In specific embodiments of any of the above-mentioned combination treatments involving compound of formula (I) and TMZ, such as items k) to n) with any number of cycles, or the embodiments with the additional WEE1 inhibitor treatment days with any number of cycles, the glioma has recurred after an initial glioma treatment. In some aspects, the initial glioma treatment is TMZ-based. The glioma may also have recurred after a maximum of 2 prior glioma treatments, one of which is TMZ-based.

In more specific embodiments, the recurrent glioma is Grade 4 as per WHO 2021. In the same or other specific embodiments, the recurrent glioma is IDH-wildtype.

In alternative more specific embodiments, the recurrent glioma is Grade 3 as per WHO 2021. In the same or other specific embodiments, the recurrent glioma is IDH-mutant.

As indicated above, in some aspects, the administration schedules described herein, for example when involving radiotherapy, may be understood to cover administration schedules which have been interrupted for one or several days (consecutive or not).

In some embodiments of the present invention, the combination of the compound of formula (I) with either TMZ or radiotherapy, or with both, shows a synergistic effect in treating glioma.

Particularly, in some embodiments, the combination of compound of formula (I) with either TMZ or radiotherapy shows a synergistic effect in treating glioma, along with an acceptable safety profile. Also, in some embodiments, the triple combination of compound of formula (I) with TMZ and radiotherapy shows a synergistic effect in treating glioma, along with an acceptable safety profile.

### Pharmaceutical Compositions

Pharmaceutical compositions of the present invention that are suitable for oral administration (oral dosage forms) may be presented in solid or liquid form. Suitable solid oral dosage forms include capsules, tablets, powders or granules and the like, each containing a predetermined amount of the active ingredient. Suitable liquid oral dosage forms include solutions, emulsions or suspensions. Pharmaceutical compositions of the present invention may also be in the form of sustained release formulations.

Any inert ingredient that is commonly used as a carrier or diluent may be used as pharmaceutically acceptable excipient in the solid oral formulations of the present invention, such as for example, a gum, a starch, a sugar, a cellulosic material, an acrylate, or mixtures thereof. Preferred diluents include, for example, microcrystalline cellulose, anhydrous lactose. The compositions may further comprise a disintegrating agent (e.g., croscarmellose sodium, sodium starch glycolate) and a lubricant (e.g., magnesium stearate), and may additionally comprise one or more additives selected from a binder (e.g., hydroxypropylcellulose), a glidant (e.g., silicon dioxide), a buffer (e.g., citric acid), a surfactant (e.g., tween 80), a solubilizing agent (e.g., cyclodextrin), a plasticizer (e.g., triacetin), an emulsifier (e.g, sodium lauryl sulfate), a stabilizing agent (e.g., povidone, ascorbic acid), a viscosity increasing agent (e.g., hydroxypropyl methylcellulose), a sweetener (e.g., sucrose), a film forming agent (e.g., cellulose based systems, polymers), a colorant (e.g., iron oxide), a flavoring agent or any combination thereof.

The oral pharmaceutical compositions of the present invention may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent.

### Compound of Formula (I)

In some embodiments of the present invention, the compound of formula (I) is formulated in a pharmaceutical composition being an oral dosage form. In more specific embodiments, the compound of formula (I) is in the form of a solid oral dosage form, such as tablets or capsules (which may comprise powder, granulate or minitablets). Capsules may be soft or hard capsules.

For example, the compound of formula (I) is formulated in capsules, such as hard gelatin capsules comprising minitablets. Each minitablet may contain 10 mg of active ingredient. The following capsule strengths may be available: 20, 30, 60, 100, 130, 150 and 200 mg of active ingredient (free base). Intermediate capsule strengths may readily be produced to cover alternative dose levels.

In some embodiments, the solid oral dosage form is packaged in a blister and/or a bottle. In some embodiments, one, two, three, four, five or six unit dosage forms are administered per intake of the compound of formula (I). Preferably, one to four unit dosage form(s) is administered per intake of the compound of formula (I).

For example, when formulated as capsules containing minitablets, the excipients in each minitablet may include microcrystalline cellulose, anhydrous lactose, hydroxypropylcellulose, sodium starch glycolate, anhydrous colloidal silica and/or magnesium stearate. Alternative excipients may be used in an oral dosage form being bioequivalent.

### Temozolomide

In some embodiments of the present invention, TMZ is formulated in a pharmaceutical composition being an oral dosage form. In more specific embodiments, the pharmaceutical composition containing TMZ is a capsule, such as a hard capsule.

The TMZ capsules may contain: lactose, lactose anhydrous, colloidal silicon dioxide, sodium starch glycolate, tartaric acid, and/or stearic acid. Alternative excipients may be used in an oral dosage form being bioequivalent.

Each capsule for oral use may contain 5 to 250 mg temozolomide, such as 5 mg, 20 mg, 100 mg, 140 mg, 180 mg, or 250 mg of temozolomide.

The inactive ingredients for TMZ capsules may, for example, be as follows: TMZ 5 mg: lactose anhydrous (132.8 mg), colloidal silicon dioxide (0.2 mg), sodium starch glycolate (7.5 mg), tartaric acid (1.5 mg), and stearic acid (3 mg). TMZ 20 mg: lactose anhydrous (182.2 mg), colloidal silicon dioxide (0.2 mg), sodium starch glycolate (11 mg), tartaric acid (2.2 mg), and stearic acid (4.4 mg). TMZ 100 mg: lactose anhydrous (175.7 mg), colloidal silicon dioxide (0.3 mg), sodium starch glycolate (15 mg), tartaric acid (3 mg), and stearic acid (6 mg). TMZ 140 mg: lactose anhydrous (246 mg), colloidal silicon dioxide (0.4 mg), sodium starch glycolate (21 mg), tartaric acid (4.2 mg), and stearic acid (8.4 mg). TMZ 180 mg: lactose anhydrous (316.3 mg), colloidal silicon dioxide (0.5 mg), sodium starch glycolate (27 mg), tartaric acid (5.4 mg), and stearic acid (10.8 mg). TMZ 250 mg: lactose anhydrous (154.3 mg), colloidal silicon dioxide (0.7 mg), sodium starch glycolate (22.5 mg), tartaric acid (9 mg), and stearic acid (13.5 mg).

For each dosage form and strength, alternative excipients may be used in an oral dosage form being bioequivalent. For example, each of the above capsule strengths may contain lactose, sodium starch glycolate (Type B), tartaric acid and stearic acid as excipients.

The body of the capsules may for example be made of gelatin. The cap may also be made of gelatin, and the color of the capsule cap may vary based on the dosage strength. The capsule body and cap may be imprinted with pharmaceutical branding ink, which may contain shellac, dehydrated alcohol, isopropyl alcohol, butyl alcohol, propylene glycol, purified water, strong ammonia solution, potassium hydroxide, and/or ferric or iron oxide.

In alternative embodiments of the present invention, TMZ is formulated in a pharmaceutical composition for injection. For example, TMZ is formulated as a powder for injection, comprising 5 to 250 mg of temozolomide, such as a 100-mg powder for injection.

As an example, a vial may contain 100 mg of sterile and pyrogen-free temozolomide lyophilized powder for intravenous injection. The inactive ingredients may be: mannitol (600 mg), L-threonine (160 mg), polysorbate 80 (120 mg), sodium citrate dihydrate (235 mg), and hydrochloric acid (160 mg). For each dosage form and strength, alternative excipients may be used in an IV dosage form being bioequivalent.

In some embodiments of the present invention, an oral dosage form, such as a solid oral dosage form, may incorporate both compound of formula (I) and TMZ as active ingredients.

Of course, in some embodiments, TMZ may be used in the form of a pharmaceutically acceptable salt. In such embodiments, etoposide indications of dosages, amounts or concentrations herein, which are indications for the free base, may be suitably adapted in view of the molecular weight of the respective salt.

Embodiments of the present disclosure can be further defined and illustrated by reference to the following non-limiting examples. It will be apparent to those skilled in the art that many modifications or changes, e.g. to the materials and methods described, can be practiced without departing from the scope of the present disclosure.

### EXAMPLES

### Example 1: Determination of Compound of Formula (I), AZD1775 and Zn-c3 Concentrations in Healthy Mouse Brain Tissue Following Repeated Administration

Healthy male Nu/Nu mice were treated orally with either (i) a single dose of 30 mg/kg Compound of formula (I) (25 mice in total, 3 mice per time-point), (ii) 30 mg/kg of Compound of formula (I) given once a day for four consecutive days (27 mice in total, 3 mice per time-point), (iii) 30 mg/kg of AZD-1775 (adavosertib, purchased from Combi-Blocks, San Diego, USA) given twice a day for three consecutive days and once on day four (27 mice in total, 3 mice per time-point), (iv) or 80 mg/kg of Zn-c3 (purchased from EOS Med Chem, Jina City, China) given once a day for four consecutive days (27 mice in total, 3 mice per time-point). The doses used for each compound have previously been reported as active dose levels. Compound of formula (I) was formulated as a suspension in 0.5% methylcellulose with 1% Tween 80 and administered at a concentration of 3.0 mg/mL. AZD-1775 was formulated as a suspension in 0.5% methylcellulose in water at a dose concentration of 3.0 mg/mL and Zn-c3 was prepared in 20% HP-β-CD (2-hydroxypropyl)-β-cyclo-dextrin) in water and administered as a solution at a dose concentration of 8 mg/mL. The dosing volume of all formulations was 10 mL/kg.

At designated time-points the mice were euthanized by carbon dioxide inhalation, blood samples were collected by cardiac puncture into K₂EDTA containing tubes. The tubes were inverted several times, placed on ice and centrifuged within 30 min of collection (4° C. for 5 min at 6000 rpm) to obtain plasma. The plasma samples were snap frozen in dry ice and stored at −80° C. until analysis. The brain was collected, weighed and snap frozen in dry ice until further analysis. To obtain brain homogenates, firstly water was added to each brain (brain weight:water volume=1:4) then the brain was homogenized with a H-speed dispersator (time procedure: homogenizing for 10 s and interval of 10 s per time, total 3-5 times; cleaning procedure for dispersator: Water-Methanol-Ethanol-Water, 10 s each). The brain homogenate samples were transferred to individual tubes and stored at −80° C. prior to analysis.

The mouse plasma and brain samples were analysed as follows. For each analytical batch of plasma and brain samples, the standard and quality control (QC) samples were freshly prepared in blank male Nu/Nu mouse plasma (with K2EDTA anticoagulant) and brain homogenate. To an aliquot of each standard, QC, control blank and study samples, internal standard (¹³CD₆-Compound of formula (I) for Compound of formula (I), terfenadine for AZD-1775 and Zn-c3) in methanol/acetonitrile (1:1, v/v) was added. The samples were mixed for 1 minute and centrifuged at 4000 rpm at 4° C. for 15 minutes. Supernatants were transferred and diluted in MeOH/water (1:1, v/v, with 0.1% FA) prior to LC-MS/MS injection.

Chromatographic separation was achieved on an Shimadzu LC-20AD using a Kinetex 2.6μ C18 100A column (50 mm*3.00 mm) for Compound of formula (I) and AZD-1775 samples and a ACE 5 C4 column (50 mm*2.1 mm) for Zn-c3 samples with gradient elution using the following conditions: flow rate: 0.7 mL/min; run time: 3.0 min; mobile phase: A: 5 mM NH4OAc (0.1% FA), B: Acetonitrile (0.1% FA).

Detection was accomplished with an AB Sciex 5500 utilizing electrospray MS/MS in positive ion multiple reaction monitoring mode (MRM). The MRM channels were 511.29/98 for Compound of formula (I), 501.47/442.20 for AZD-1775 and 527.36/468.10 for Zn-c3. The MRM channels of internal standard were 518.17/102.10 (¹³CD₆-Compound of formula (I)) for Compound of formula (I), 472.40/436.40 (terfenadine) for AZD-1775 and Zn-c3.

The measured plasma and brain concentrations at each time-point, the nominal sample collection time, and intended dosages were used for pharmacokinetic analysis. The PK parameters of all study compounds were determined by non-compartmental analysis using WinNonlin Version 8.0 (Pharsight). The results are summarized in Table 2 below.

TABLE 2

| | Test Item | | | | | | | |
|---|---|---|---|---|---|---|---|---|
| | Compound of formula (I) | | | | AZD-1775 | | Zn-c3 | |
| | Dose | | | | | | | |
| | | | | | 30 mg/kg, bid for | | | |
| | 30 mg/kg, single | | 30 mg/kg, qd for | | 3 days and once | | 80 mg/kg qd for | |
| | dose | | four days | | on day 4 | | four days | |
| | Time-points for PK sampling | | | | | | | |
| | 0.5, 1, 2, 4, 8, | | pre-dose, 0.5, 1, | | pre-dose, 0.5, 1, | | pre-dose, 0.5, 1, | |
| | 24, 32, 48 hr | | 2, 4, 8, 24, 32, | | 2, 4, 8, 12, 24, | | 2, 4, 8, 24, 32, | |
| | post dose on | | 48 hr post dose | | 32 hr post dose | | 48 hr post dose | |
| | day 1 | | on day 4 | | on day 4 | | on day 4 | |
| | Matrix | | | | | | | |
| | plasma | brain | plasma | brain | plasma | brain | plasma | brain |
| Cₘₐₓ (ng/mL for | 7037 | 3557 | 3943 | 1963 | 1260 | 53.9 | 2773 | 97.2 |
| Plasma; ng/g for | | | | | | | | |
| Brain) | | | | | | | | |
| AUC_{Last} (hr*ng/mL | 85146 | 37368 | 64825 | 31610 | 1953 | 94.5 | 14179 | 396 |
| for Plasma; hr*ng/g | | | | | | | | |
| for Brain) | | | | | | | | |
| AUC_{Inf} (hr*ng/mL for | 85218 | 37471 | 64953 | 31763 | 1958 | 99.7 | 14180 | 878 |
| Plasma, hr*ng/g for | | | | | | | | |
| Brain) | | | | | | | | |

| | | | | |
|---|---|---|---|---|
| Brain to plasma ratio | 0.505 | 0.498 | 0.043 | 0.035 |
| based on Cₘₐₓ | | | | |
| Brain to plasma ratio | 0.439 | 0.488 | 0.048 | 0.028 |
| based on AUC_{Last} | | | | |
| Brain to plasma ratio | 0.440 | 0.489 | 0.051 | 0.062 |
| based on AUC_{Inf} | | | | |

AUC = Area under the concentration-time curve, measuring the exposure of the test items (Compound of formula (I), AZD-1775, Zn-c3) in plasma or brain.
Cmax = Maximum concentration of the test item measured in plasma or brain.
AUClast = Area under the concentration-time curve, based on time 0 to the last measured concentration in brain or plasma. For example, for Compound of formula (I), the last measured time point in plasma was 48 hrs and the AUClast reflects the area under the plasma concentration-time curve from 0 hrs to 48 hrs.
AUCinf = Area under the concentration-time curve, based on time 0 and extrapolated to infinite time (i.e. beyond the last measured time-point). For example, for Compound of formula (I), the last measured time point in plasma was 48 hrs and the AUCinf reflects the exposure from time 0 hrs to beyond 48 hrs.

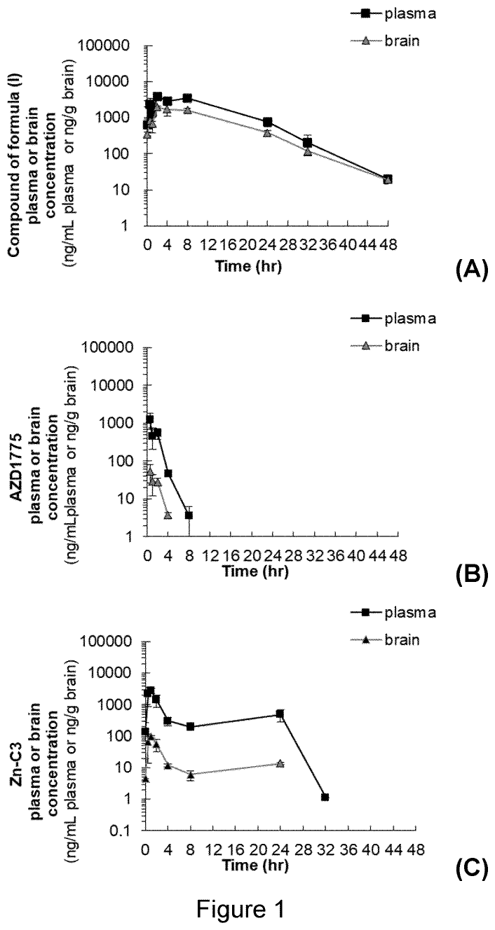

FIG. 1 shows 3 graphs representing the plasma and brain concentrations measured on day 4, following repeated dose administration of 30 mg/kg qd of Compound of formula (I) (FIG. 1(A)), 30 mg/kg bid of AZD1775 (FIG. 1(B)) and 80 mg/kg qd of Zn-c3 (FIG. 1(C)).

The results of Example 1 demonstrate that Compound of formula (I) can cross the blood brain barrier (BBB) and distributes into the brain. Furthermore, the data shows that the brain distribution properties of Compound of formula (I) are favourable compared to AZD1775 and Zn-c3, which is reflected by the higher brain to plasma exposure ratios of Compound of formula (I) (more than 10-fold higher than AZD1775; more than 7-fold higher than Zn-C3).

### Example 2: Determination of Compound of Formula (I) Concentrations in Healthy Rat Brain Tissue and Cerebrospinal Fluid Following Single Administration

Healthy male Wistar Han rats were treated orally with a single dose of 15 mg/kg Compound of formula (I) (24 rats in total, 3 rats per time-point). Compound of formula (I) was formulated as a suspension in 0.5% methylcellulose with 1% Tween 80 and administered at a concentration of 1.5 mg/mL with a dosing volume of 10 mL/kg.

At designated time-points (0.5; 1; 2; 4; 8; 24; 32; 48 hrs post-dose), the rats were euthanized by carbon dioxide inhalation. Thereafter, blood, CSF, and brain samples were collected as follows. Blood samples were collected by cardiac puncture into K₂EDTA containing tubes. The tubes were inverted several times, placed on ice and centrifuged within 30 min of collection (4° C. for 5 min at 6000 rpm) to obtain plasma. The plasma samples were snap frozen in dry ice and stored at −80° C. until analysis. The CSF samples were collected by using a scalp-needle to puncture the foramen magnum. The CSF samples were acidified with 10% formic acid (final sample concentration of 0.5% formic acid) and stored in −80° C. until further analysis. The brain was collected, weighed and snap frozen in dry ice until further analysis. To obtain brain homogenates, firstly water was added to each brain (brain weight:water volume=1:4) then the brain was homogenized with a H-speed dispersator (time procedure: homogenizing for 10 s and interval of 10 s per time, total 3-5 times; cleaning procedure for dispersator: Water-Methanol-Ethanol-Water, 10 s each). The brain homogenate samples were transferred to individual tubes and stored at −80° C. prior to analysis.

The samples were analyzed as follows. For each analytical batch of plasma and brain samples, the standard and quality control (QC) samples were freshly prepared in blank male Wistar Han plasma (with K₂EDTA anticoagulant) and brain homogenate. Acidified (containing 0.5% formic acid) artificial CSF was used as standard and quality control for the CSF samples. Additionally, one set of blank male Wistar Han CSF was included as QC sample.

To an aliquot of each standard, QC, control blank and study samples, internal standard (¹³CD₆-Compound of formula (I)) in methanol/acetonitrile (1:1, v/v) was added. The samples were mixed for 1 minute and centrifuged at 4000 rpm at 4° C. for 15 minutes. Supernatants were transferred and diluted in MeOH/water (1:1, v/v, with 0.1% FA) prior to LC-MS/MS injection.

Chromatographic separation was achieved on a Shimadzu LC-20AD (for plasma and brain samples) or Shimadzu LC-30AD (for CSF samples) using a Kinetex 2.6μ C18 100A column (50 mm*3.00 mm; for plasma and brain samples) or an ACE 5 C4 column (50 mm*2.1 mm; for CSF samples). The following conditions were applied: flow rate: 0.7 mL/min; run time: 3.0 min; mobile phase: A: 5 mM NH₄OAc (0.1% FA), B: Acetonitrile (0.1% FA).

Detection was accomplished with an AB Sciex 5500 utilizing electrospray MS/MS in positive ion multiple reaction monitoring mode (MRM). The MRM channels were 511.29/98 for Compound of formula (I) and 518.17/102.10 for the internal standard ¹³CD₆-Compound of formula (I).

The measured plasma, CSF and brain concentrations at each time-point, the nominal sample collection time, and intended dosages were used for pharmacokinetic analysis. The PK parameters of all study compounds were determined by non-compartmental analysis using WinNonlin Version 8.0 (Pharsight). The results are summarized in Table 3 below.

TABLE 3

| | Dose of compound of formula (I) | | |
|---|---|---|---|
| | 15 mg/kg p.o. single dose | | |
| Matrix | plasma | brain | CSF |
| Cₘₐₓ (ng/mL for Plasma | 1138 | 588 | 15.6 |
| or CSF; ng/g for Brain) | | | |
| AUC_{Last} (hr*ng/mL for Plasma | 14057 | 8383 | 219 |
| or CSF; hr*ng/g for Brain) | | | |
| AUC_{Inf} (hr*ng/mL for Plasma | 14082 | 8415 | 221 |
| or CSF; hr*ng/g for Brain) | | | |
| Brain to plasma or CSF to unbound | n.a. | 0.52 | 0.18* |
| plasma ratio based on Cₘₐₓ | | | |
| Brain to plasma or CSF to unbound | n.a | 0.60 | 0.20* |
| plasma ratio based on AUC_{Last} | | | |
| Brain to plasma or CSF to unbound | n.a | 0.60 | 0.20* |
| plasma ratio based on AUC_{Inf} | | | |

*Compound of formula (I) concentrations in CSF reflect unbound drug concentrations. Thus, to determine the CSF to plasma ratios, the unbound plasma concentrations are more relevant than total concentrations. Unbound Compound of formula (I) plasma concentrations were calculated based on a free fraction of 7.8% determined in rat plasma in a separate experiment (not shown), as follows. For example, Cmax plasma (1138 ng/mL) * free fraction in rat plasma (0.078) = Unbound plasma concentration (88.76 ng/mL). Consequently, the CSF to unbound plasma ratio was calculated as follows: CSF concentration (15.6 ng/mL)/Unbound plasma concentration (88.76 ng/mL) = 0.18.

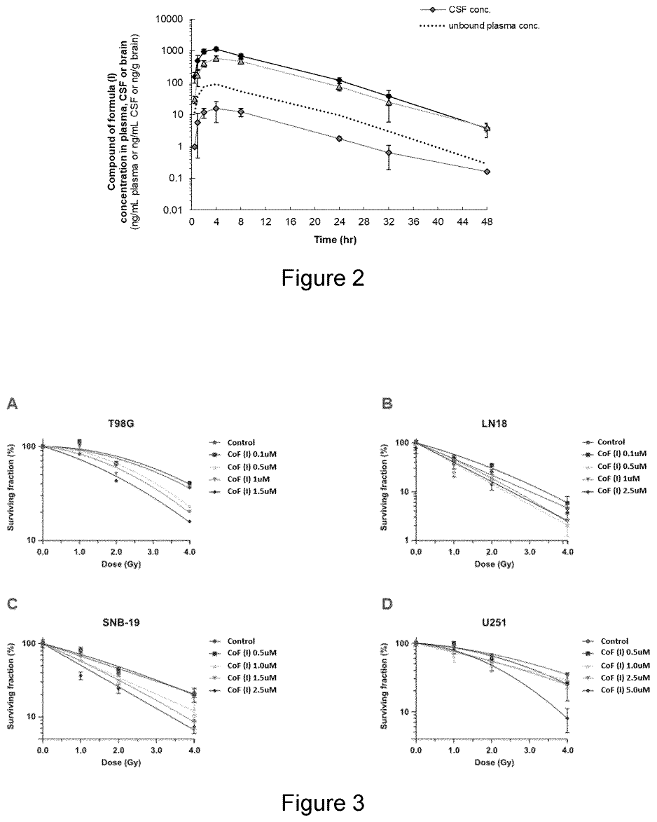

FIG. 2 graphically shows the total plasma concentration, total brain concentration, unbound CSF concentration and calculated unbound plasma concentration of Compound of formula (I) over time.

The results of Example 2 demonstrate that in rats, the compound of formula (I) is able to cross the blood brain barrier and distributes in the brain. Furthermore, the results shows that compound of formula (I) crosses the blood-CSF barrier and is able to distribute in the CSF.

### Example 3: Determination of Drug Concentration in Monkey Brain Following Daily Oral Administration for 91 Days

Compound of formula (I) was orally given to 3 female cynomolgus monkeys by gavage once a day for 91 days. The volume of administration was 5 mL/kg, and the vehicle was an aqueous solution of 0.5% (w/v) methylcellulose (cps 400) and 1% (w/v) Tween 80. The Compound of formula (I) dose was 10 mg/kg; this dose level was assessed in this study to be the minimum no adverse effect level (NOAEL) based on general toxicology evaluation. The animals were necropsied 24 hours after the last dose after an overnight period without food. A sample from brain tissue (left forebrain) was collected from each animal, snap frozen in liquid nitrogen. and stored frozen at <−50° C. Blood samples were taken from the femoral vein into tubes containing K₂EDTA, fully inverted approximately 10 times, and placed on crushed wet ice until centrifugation at 2300 g for 10 minutes at approximately 4° C. The resultant plasma was separated, transferred into polypropylene tubes, and frozen at <−50° C. Tissue and plasma samples were transported frozen on dry ice to the respective sites of analysis.

The monkey plasma samples were analyzed for their contents in Compound of formula (I) and N³²-desmethyl-Compound of formula (I) as follows. The plasma samples were fortified in human K₂-EDTA plasma (1:9 monkey:human volume ratio) prior to extraction. All solution preparations and dilutions were conducted under sodium vapor light and in “low-binding” quality polypropylene tubes and deep-well plates. The analytes and internal standards (¹³CD₆-Compound of formula (I) and D₇-N³²-desmethyl-Compound of formula (I)) were extracted by protein precipitation with 5M aqueous urea (40 μL for 50 μL plasma), and then analyzed by LC-MS/MS. Chromatographic separation was achieved on an Synergi Polar-RP, 80A, 4 μm 75×2.00 mm column (Phenomenex) with gradient elution using a mixture of 0.1% formic acid in water as eluent A and 0.1% formic acid in acetonitrile as eluent B (5% B from 0 to 0.50 min, 42% B at 4.5 min, 95% B from 4.60 min to 5.50 min, 5% B at 5.60 min, 100% B at 5.70 min, 5% B at 6.40 min, 100% B at 6.50 min, 5% B at 7.00 min; 800 μl/min, 70° C.). Detection was accomplished with a AB Sciex 6500+ mass spectrometer utilizing electrospray MS/MS in positive ion multiple reaction monitoring mode (MRM). The lower limits of quantification for Compound of formula (I) and N³²-desmethyl-Compound of formula (I) were 5.00 ng/mL and 2.50 ng/mL, respectively.

The monkey brain tissue samples were analyzed for their contents in Compound of formula (I) and N³²-desmethyl-Compound of formula (I) as follows. Tissue samples were homogenized in human plasma (1 mg tissue for 10 μL plasma) at +4° C. using a Precelly Evolution system. The analytes (Compound of formula (I) and N³²-desmethyl-Compound of formula (I)) were extracted by protein precipitation with methanol/acetonitrile (9:1, v:v) containing the internal standards (¹³CD₆-Compound of formula (I) and D₇-N³²-desmethyl-Compound of formula (I)) and then analyzed by UHPLC-MS/MS. Chromatographic separation was achieved on a HSS T3 1.8 μm, 2.1×100 mme column (Waters) with gradient elution using a mixture of 0.1% formic acid in water as eluent A, and 0.1% formic acid in acetonitrile as eluent B (20% B from 0 to 0.50 min, 90% B from 1.80 min to 2.30 min, 20% B at 2.60 min; 0.6 mL/min, room temperature). Detection was accomplished with Xevo TQ-S mass spectrometer (Waters) utilizing electrospray MS/MS in positive ion multiple reaction monitoring mode (MRM). The lower limits of quantification for Compound of formula (I) and N³²-desmethyl-Compound of formula (I) were 55.00 ng/g tissue and 22.00 ng/g tissue, respectively.

The results are shown in Table 4 below.

TABLE 4

| | Plasma | Brain | | Mean |
|---|---|---|---|---|
| | concentration | concentration | Brain:plasma | brain:plasma |
| | at 24 h post | at 24 h post | concentration | ratio (±SD), |
| | last dose | last dose | ratio, at 24 h | at 24 h post |
| | (ng/mL) | (ng/g tissue) | post last dose | last dose (n = 3) |
| Compound of | 929 | 3885 | 4.18 | 4.32 ± 0.22 |
| formula (I) | 632 | 2886 | 4.57 | |
| | 1140 | 4786 | 4.20 | |
| N³²-Desmethyl- | 531 | 258 | 0.49 | 0.73 ± 0.21 |
| Compound of | 233 | 188 | 0.81 | |
| formula (I) | 365 | 323 | 0.88 | |

For both Compound of formula (I) and its N³²-desmethyl metabolite, at 24 h post last dose, the concentration in the brain is not negligible compared to the plasma concentration, which shows that both compounds have the ability to cross the blood-brain barrier.

### Example 4: In Vitro Efficacy of Compound of Formula (I) in Combination with Radiotherapy (RT) in GBM Cell Lines

GBM tumor cells (T98G, LN18, SNB-19 and U251) were grown as an adherent monolayer at 37° C. in a humidified atmosphere (5% CO2, 95% air). For experimental use, tumor cells were detached from culture flasks by a 5-minute treatment with trypsin-versene, in Hanks' medium without calcium or magnesium and neutralized by addition of complete culture medium. Cells were counted and viability assessed using a 0.25% trypan blue exclusion assay. All cell lines were appropriately seeded following the conditions for monolayer colony formation in 6-well plates (Ref #152795, Nunc, Dutscher) in 1.9 ml of cell culture medium, at 3 densities for evaluation of a proclonogenic effect. Cell densities were 500, 100 or 200 cells per well for GL261 and LN-18 cell lines, 1500, 2000 and 2500 cells per well for SNB-19 cell line and 250, 500 and 1000 cells per well for the T98G cell line.

Treatments were performed at 24 hours (D1) after seeding by addition of ×20 concentrated compound or vehicle solutions, to obtain final concentrations of 0.1 μM to 5.0 μM of Compound of formula (I) [referred to as CoF (I) in FIG. 3]. A 0.1% DMSO vehicle was added as control. Immediately following compound treatment, the plates were irradiated at 1, 2 and 4 Gy. The irradiation was performed with SARRP irradiation system (Xstrahl life science). Mock irradiation was performed on the plate corresponding to non-irradiated control. At Day 2, the day after treatment and irradiation, and day 7, culture medium was replaced in all plates with fresh medium without Compound of formula (I). After 14 days of incubation, colonies were counted after being stained through incubation with 500 μl of a solution of water containing 0.05% (w/v) of methylene blue (Ref. M4159, Sigma) for 2 hours. Colonies were counted using a colony counter pen (Ref #045855, Dutscher, Brumath, France). The Surviving Fraction was calculated for each condition using the equations described below and plotted on a semi-logarithmic graph using the Linear-Quadratic model. Weighing of the curve was done using 1/y². Statistical testing was done using an extra sum-of-squares F test and H₀=one curve accurately fits all data. All analyses were done using GraphPad Prism 7 (Graphpad Inc., La Jolla, CA)

$$Plating efficiency = \frac{Number of colonies counted}{Number of cells seeded} * 100$$

$$Surviving Fraction = \frac{Plating efficiency of treated condition}{Plating efficiency of control ( untreated )} * 100$$

$$Linear Quadratic Model : Surviving Fraction = 100 * {e}^{- ( α * Dose ) - ( β * {Dose}^{2} )}$$

Experiments were pooled together per cell line for further analysis using LinearQuadratic model.

The results show that compound of formula (I) significantly increased effect of radiotherapy in T98G, SNB19 and LN18 cell lines (p<0.0001) (FIG. 3). This was not significant in case of U251 cell line (p=0.6); however based on simulation, running the same analysis with more replicates or by comparing only the control and 5 μM conditions alone would have been statistically significant for this cell line as well.

### Example 5: In Vivo Efficacy of Compound of Formula (I) as a Monotherapy in the U87-MG-Luc GBM Cell Line

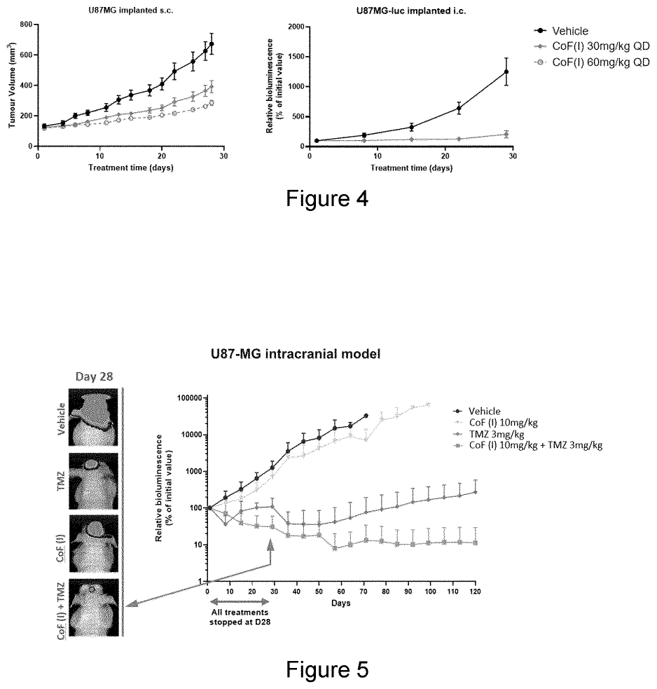

Male athymic nude mice were injected A) subcutaneously (s.c.) or B) cerebrally (i.e.) with 1×10⁵ U87-MG-luc cells in Matrigel. Animals implanted i.e. were imaged to monitor tumor establishment and growth throughout the study using an In Vivo Imaging System (IVIS) once weekly, detecting tumor bioluminescence (BLI) in photons. For animals implanted s.c., tumor growth was monitored twice weekly by caliper measurement and tumor volume calculated using the equation V=(L×W×W)/2, where V is tumor volume, L is tumor length (the longest tumor dimension) and W is tumor width (the longest tumor dimension perpendicular to L). Upon confirmation of tumor establishment, animals were randomly allocated into 3 (A, s.c.) or 2 (B, i.e.) treatment groups (n=8 animals per group) consisting of vehicle (0.5% methycellulose, 1% Tween 80 P.O. and saline I.V.) or Compound of formula (I) [CoF (I)] formulated as a suspension in 0.5% methycellulose and 1% Tween 80. The treatments were administered over a 28 day cycle and compound of formula (I) was administered orally at a dose of either 30 mg/kg (for both A and B) or 60 mg/kg (for A only), once daily (QD)). During routine monitoring, the animals were checked for any effects of tumor growth and treatments on behavior such as mobility, food and water consumption, body weight gain/loss 3 times a week, eye/hair matting and any other abnormalities. Mortality and observed clinical signs were recorded for individual animals in detail. The results are shown in FIG. 4.

Treatment of subcutaneously implanted U876-MG-luc tumors with Compound of formula (I) at either 30 mg/kg or 60 mg/kg resulted in significantly smaller tumors than vehicle controls at the end of the treatment period (p=0.0009 and p<0.0001 respectively on day 28).

Treatment of cerebrally implanted U876-MG-luc tumors with Compound of formula (I) at 30 mg/kg resulted in significantly smaller tumors than vehicle controls at the end of the treatment period (p=0.0023 on day 28).

No significant body weight loss was observed throughout the study, indicating that treatments with Compound of formula (I) at all dose levels were well tolerated.

Thus, compound of formula (I) used in monotherapy for treatment of GBM tumors implanted either subcutaneously or cerebrally was well tolerated and resulted in significant anti-tumor activity.

### Example 6: In Vivo Efficacy of Compound of Formula (I) in Combination with Temozolomide in the U87-MG-Luc GBM Cell Line

Male athymic nude mice were injected into the cerebellum with 1×10⁵ U87-MG-luc cells in Matrigel. Animals were imaged to monitor tumor establishment and growth throughout the study using an In Vivo Imaging System (IVIS) once weekly, detecting tumor bioluminescence (BLI) in photons. Once tumor establishment was confirmed, animals were randomly allocated into 8 treatment groups: vehicle (0.5% methycellulose, 1% Tween 80 P.O. and saline IV.), Compound of formula (I) [CoF (I)](formulated as a suspension in 0.5% methycellulose and 1% Tween 80) administered orally at 3 mg/kg, at 10 mg/kg or at 30 mg/kg once daily (QD) in monotherapy, temolozomide [TMZ] administered orally at 3 mg/kg once daily (QD) in monotherapy, over a 28 day cycle, and combination of TMZ+compound of formula (I) at each dose) (n=6 animals per group for monotherapy groups and n=8 animals per group for combination groups). In combination arms, Compound of formula (I) and was administered 2 hours prior to TMZ. During routine monitoring, the animals were checked for any effects of tumor growth and treatments on behavior such as mobility, food and water consumption, body weight gain/loss 3 times a week, eye/hair matting and any other abnormalities. Mortality and observed clinical signs were recorded for individual animals in detail.

Treatment of cerebrally implanted U876-MG-luc tumors with Compound of formula (I) at 3 mg/kg or 10 mg/kg as a monotherapy did not result in any significant anti-tumor activity as determined by mean relative BLI, whereas treatment with Compound of formula (I) at 30 mg/kg resulted in significantly smaller tumors than vehicle controls (p=0.0133 on day 36), consistent with results presented in Example 5 (B i.e.). As U876-MG-luc is a TMZ-sensitive tumor model, treatment with TMZ resulted in significant anti-tumor response (p=0.0066 vs vehicle on day 36) which persisted throughout the monitoring period with 3 out of 6 animals treated experiencing complete responses.

Furthermore, treatment of U87MG-luc tumors with Compound of formula (I) in combination with TMZ also led to a significant anti-tumor activity compared to vehicle controls (p<0.001 at all doses of Compound of formula (I) at day 28) which persisted for up to 160 days. Additionally, combination treatments at the 10 mg/kg and 30 mg/kg doses of Compound of formula (I) showed significant anti-tumor activity compared to standard of care TMZ treatment during the treatment period (p=0.0213 and p=0.0174 respectively on day 28) (FIG. 5 shows that a dose of 10 mg/kg of compound of formula (I) in combination with TMZ is sufficient to get a significant anti-tumor effect). In each combination group, treatments led to durable responses throughout the monitoring period with 3 out of 8 (3 mg/kg Compound of formula (I)), 6 out of 8 (10 mg/kg Compound of formula (I)) and 5/7 (30 mg/kg Compound of formula (I)) animals experiencing complete responses (defined as BLI value=0).

TMZ at 3 mg/kg, Compound of Formula (I) at 3, 10 and 30 mg/kg as a monotherapy and Compound of Formula (I) in combination with Temozolomide at all tested doses were also well tolerated with no significant change in animal body weights compared to pre-treatment levels over the course of the 28-day treatment period. All groups maintained their body weight after the treatment period, showing no post-treatment intolerance.

### Example 7: In Vivo Assessment of Compound of Formula (I) as a Monotherapy or in Combination with TMZ on Survival of Mice Harboring GL261 Intracranial Tumors

GL261 cells were harvested during log phase growth and resuspended in phosphate buffered saline (PBS). Female C57/BL6 mice were inoculated with 1×10⁵ GL261 cells in Matrigel (10 μL cell suspension) via intracranial injection. In accordance with the protocol, the injection site was determined based on the intersection of the midline suture and the caudal transverse suture line. The needle point of a 25-gauge needle was placed 1-mm cranial and 1-mm to either the right or left of the intersection, and the 10 μL GL261 glioma cell suspension was injected into the space. Intracranial injections were made five days prior to the beginning of treatment.

Compound of formula (I) [CoF (I)] and temozolomide were administered orally (p.o.) at a dosing volume of 10 mL/kg, scaled to the most recent body weight (BW) of the individual animals. Compound of formula (I) was administered once daily dosing for fourteen (qd×14) or twenty-eight days (qd×28). Temozolomide was administered once daily for five days (qd×5). For combination therapy groups, Compound of formula (I) was administered 2 h prior to temozolomide administration. Five days after GL261 tumor cell implantation, designated as Day 1 of the study, mice were sorted by BW into eight groups (n=8). Dosing was initiated on Day 1, Group 1 mice received vehicle p.o. qd×14 and served as the control for efficacy analysis. Group 2 received Compound of formula (I) at 30 mg/kg p.o. qd×14, Group 3 temozolomide at 3 mg/kg qd×5 and group 4 mice Compound of formula (I) at 30 mg/kg qd×14 in combination with temozolomide at 3 mg/kg qd×5. The study endpoint was death or moribundity due to glioma progression or the last day of the study, whichever came first (i.e. event). The median time to event (TTE) value was calculated for each group. The median TTE of treated mice was expressed as a percentage of the median TTE of the control mice (% T/C), and the increase in life span (ILS) was calculated as: ILS=% T/C−100%, where T=median TTEtreated, and C=median TTEcontrol. Thus, if T=C, ILS=0%. The maximum % ILS for this study, based on the ratio between the study endpoint and the median TTE of the control group (22.5 days), is 296% (Teicher, Beverly A. Tumor models in cancer research. New York: Humana Press Springer, 2011).

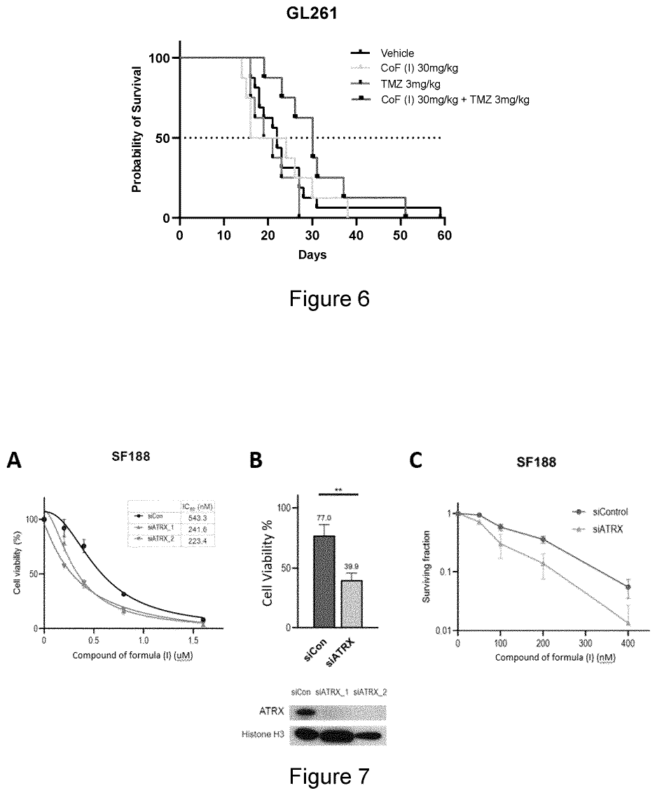

For the combination group of Compound of formula (I) at 30 mg/kg and temozolomide the median TTE was 30.0 days, corresponding to 33% ILS. Overall survival in the combination group improved upon the temozolomide single agent regimen (p<0.01) (FIG. 6).

### Example 8: In Vitro Efficacy of Compound of Formula (I) as a Monotherapy in ATRX Deficient GBM Cell Lines

SF188 cells were transfected with ATRX siRNA (siATRX_1 or siATRX_2) either individually (A) or as a pool (B and C) or control siRNA (siCon) and ATRX deficiency assessed by determining relative ATRX protein levels by western blotting. Control and ATRX-deficient SF188 cells were then treated with compound of formula (I) at a range of concentrations between 0 and 1.5 uM (FIG. 7A) and at 400 nM (FIG. 7B) and drug sensitivities were assessed via an ATP-based cell viability assay or in colony formation assay.

For cell viability assessment, SF188 cells were seeded in 96-well culture plates at 100-500 cells in 100 μl of culture media per well. After overnight incubation, 100 μl of culture media containing compound of formula (I) was added to each well at the desired final concentration. After 72 h, cell viability was assessed by CellTiter-Glo 2.0 according to the manufacturer's instructions. Each condition was tested in triplicate or quadruplicate, and the average viability is presented as a percentage of vehicle-treated cells. For colony formation assessment (FIG. 7C), SF188 cells were seeded on a 6-well plate at 100 cells per well in 1 ml of culture media. After overnight incubation, 1 ml of culture media containing compound of formula (I) was added to each well at the desired final concentrations up to 400 nM. At 10 days post-treatment, cell colonies were fixed with 70% ethanol for 30 min, washed with distilled water, and stained with Coomassie Blue. The number of colonies in each well was counted manually. Each colony consisted of >50 cells (corresponds to six doublings from a single cell). Colony-forming efficiency was determined by normalizing the colony numbers to the vehicle-treated cells.

In the colony formation assay (FIG. 7C), treatment with compound of formula (I) significantly reduced the surviving fraction of cells, indicating anti-tumor activity. In cell viability assays (FIGS. 7A and 7B), treatment with compound of formula (I) significantly reduced the viability of SF188 cells with ATRX siRNA significantly improving this response to Compound of Formula (I) with an IC50 less than half of control siRNA (FIG. 7A). Cell viability of ATRX-deficient SF188 cells was significantly reduced (p<0.01) following treatment at 400 nM of compound of formula (I) (FIG. 7B). Western blotting demonstrated that treatment with siRNA ATRX pool 1 (ATRX_1) or pool 2 (ATRX_2) resulted in significantly reduced protein levels of ATRX (bottom of FIG. 7B).

### Example 9: Phase 1/2 Clinical Trial Involving Compound of Formula (I) (Referred Herein to as the Study Drug)

A clinical trial will be conducted, which is a Phase 1/2, multi-center, open-label, non-randomized, controlled study in adult participants with glioma, described as follows.

Phase 1 (dose escalation) is composed of 2 arms:
Arm A: The study population of this arm is adult participants with a confirmed diagnosis (according to WHO 2021) of
   GBM isocitrate dehydrogenase (IDH)-wildtype, Grade 4, or
   Astrocytoma, IDH-mutant, Grade 3,
who progressed or have experienced disease recurrence after a maximum of 2 prior treatment lines. All participants must have received temozolomide (TMZ) based chemoradiotherapy as first-line treatment. These participants will receive oral, once-daily dosing in 28-day cycles as follows: Study drug (escalating doses) on Day (D) 1-5, D8-10, and D15-17 and TMZ (150 mg/m²/day) on D1-5 of each cycle. Treatment will continue for a maximum of 2 years or until progression of disease, unacceptable toxicity, participant withdrawal, discontinuation as per Investigator's decision, initiation of subsequent antineoplastic treatment, the end of the study, or death, whichever occurs first. The starting dose of Study drug is 150 mg which is based on the dose levels determined to be safe in the ongoing phase 1 studies, namely the 101-study involving the Study Drug in combination with carboplatin and the 102-study involving the Study Drug in monotherapy.
Arm B: The study population of this arm is adult participants with confirmed, newly diagnosed GBM IDH-wildtype, Grade 4 (WHO 2021). These participants will receive oral, once-daily dosing for 6 weeks as follows: continuous, daily doses of Study drug (escalating doses) and TMZ (75 mg/m²/day) with concomitant administration of radiotherapy (RT; conventionally fractionated regimen of 2 Gy per fraction daily, 5 consecutive days per week, total dose 60 Gy over 30 fractions). The starting dose of Study drug is 90 mg which is based on available data from the dose levels determined to be safe in the ongoing phase 1 studies, namely the 101-study involving the Study Drug in combination with carboplatin and the 102-study involving the Study Drug in monotherapy. Emerging data, including those from Arm A of this clinical trial, will be evaluated to confirm the proposed starting dose. Following a 4-to-6-week rest period after the end of the study treatment, a maintenance treatment with TMZ for 5 days (D1-5), repeated every 28 days, with or without the addition of alternating, electric, tumor-treating fields (TTFields) (Optune®) therapy may be administered at the discretion of the Investigator as per the standard of care (SOC). Arm B will begin after at least 2 dose levels have been found to be safe in Arm A.

Dose recommendations for Study drug during the dose escalation phase will be provided independently for Arm A and Arm B. The maximum tolerated dose (MTD) and/or recommended Phase 2 dose (RP2D) to be used in Part 2 is decided based on Safety Monitoring Committee (SMC) recommendation.

Following the End of Treatment (EOT) visit, participants in Phase 1 will be followed for safety and antitumor activity until disease progression or, in case of lack of disease progression, until start of subsequent antineoplastic therapy, withdrawal of consent, loss to follow-up, death, or the end of the study, whichever occurs first.

Phase 2 will start once the RP2D of Phase 1 Arm A has been determined following recommendation by the SMC. Phase 2 will be a multi-center, open-label, externally controlled study comparing the efficacy and safety of Study drug when administered in combination with TMZ versus SOC in adult participants with a confirmed diagnosis of GBM IDH-wildtype, Grade 4 (WHO 2021), who received first-line TMZ-based chemoradiotherapy and have progressed or experienced disease recurrence. The comparison will be made using a group sequential design comparing participants assigned to treatment with Study drug and TMZ to patients in an External Control Arm (ECA).

Phase 2 is composed of 2 arms:
Experimental Arm: The study population consists of adult participants with a confirmed diagnosis of GBM IDH-wildtype, Grade 4 (WHO 2021) who received first-line TMZ-based chemoradiotherapy and have progressed or experienced disease recurrence. The participants enrolled in this arm will receive oral, once-daily dosing in 28-day cycles as follows: Study drug at the RP2D on D1-5, D8-10, and D15-17 and TMZ (150 mg/m²/day) on D1-5. Treatment will continue for a maximum of 2 years or until progression of disease, unacceptable toxicity, participant withdrawal, discontinuation as per Investigator's decision, end of the study, or death, whichever occurs first.
ECA: The study population consists of adult participants with a confirmed diagnosis of GBM IDH-wildtype, Grade 4 (WHO 2021), who received first-line TMZ-based chemoradiotherapy and have progressed or experienced disease recurrence. The ECA includes patient-level data exclusively from patients treated with SOC therapy from recently completed historical clinical trials (HCTs). Propensity score methods will be used to balance the baseline and disease-specific prognostic factors of the ECA patients to the Study drug participants.

Safety and efficacy of participants assigned to treatment with Study drug and TMZ in Phase 2 will be compared to patients in the ECA.

Following the End of Treatment (EOT) visit, participants in the Experimental Arm will be followed for safety and antitumor activity until disease progression or, in case of lack of disease progression, until start of subsequent antineoplastic therapy, withdrawal of consent, loss to follow-up, death, or the end of the study, whichever occurs first. In addition, these participants will be followed for OS.

In Phase 1 Arm A and Phase 2 Experimental Arm, a treatment cycle is defined as 28 days.

### Abbreviations Used in the Tables and Text

AE=adverse event;
AUC=area under the concentration time curve;
AUC24=area under the concentration time curve over 24 hours;
AUClast=area under the concentration time curve up to the last measurable concentration;
BOR=best overall response;
CL/F=apparent clearance;
Cmax=maximum plasma concentration;
CSF=cerebrospinal fluid;
Ctrough=trough plasma concentration;
CV=coefficient of variation;
DC=disease control;
DCR=disease control rate;
DOR=duration of response;
DLT=dose-limiting toxicity;
DOR=duration of response;
ECG=electrocardiogram;
ECHO=echocardiogram;
EOT=End of Treatment;
FACT-Br=Functional Assessment of Cancer Therapy-Brain;
GBM=glioblastoma;
Gd-based=gadolinium-based (for the contrast agent used in MRI)
ka=absorption rate constant;
KPS=Karnofsky Performance Status; KPS is an assessment tool for functional impairment. It is a standard way of measuring the ability of participants with cancer to perform ordinary tasks. The scores range from 0 (death) to 100 (no evidence of disease). A higher score means the participant is better able to carry out daily activities.
LVEF=Left ventricular ejection fraction;
mRANO=Modified Response Assessment in Neuro-Oncology; namely the recommendations set out in “Modified Criteria for Radiographic Response Assessment in Glioblastoma Clinical Trials” by Ellington B M, et al. 2017, Neurotherapeutics, 14(2):307-20.
NCI-CTCAE 5.0=National Cancer Institute Common Terminology Criteria for Adverse Events, version 5.0;
OR=objective response;
ORR=objective response rate;
OS=overall survival;
PD=progressive disease;
PFS=progression-free survival;
PK=pharmacokinetic(s);
QoL=quality of life;
RANO=Response Assessment in Neuro-Oncology; namely the recommendations set out in “Updated response assessment criteria for high-grade gliomas: response assessment in neuro-oncology working group” by Wen P Y, et al. 2010, J Clin Oncol 28(11): 1963-72.
RP2D=recommended Phase 2 dose;
RT=radiotherapy;
SOC=standard of care;
t½=apparent terminal half-life;
TEAE=treatment-emergent adverse event;
tmax=time to Cmax;
TMZ=temozolomide;
Vd=volume of distribution;
Vd/F=apparent Vd

The Primary Objectives and Estimands (including endpoints) of the clinical trial include:

| Objectives | Estimands | Ref. # |
|---|---|---|
| Primary Objectives | | |
| Phase 1 - Dose escalation - | | |
| Arm A and B | | |
| To identify the RP2D of | Endpoint: Occurrence of dose-limiting toxicity (DLT) | 1 |
| Study drug in combination | Population: | |
| with TMZ (Arm A) and in | Arm A: Adult participants with a confirmed | |
| combination with TMZ and | diagnosis of GBM IDH-wildtype, Grade 4, or | |
| RT (Arm B) and to | astrocytoma, IDH-mutant, Grade 3, who | |
| characterize the safety and | progressed, or have experienced disease | |
| tolerability of these | recurrence after a maximum of 2 prior treatment | |
| combinations. | lines and who received TMZ-based | |
| | chemoradiotherapy as first-line treatment. | |
| | Arm B: Adult participants with confirmed newly | |
| | diagnosed GBM IDH-wildtype, Grade 4 | |
| | Treatment: Study drug in combination with TMZ | |
| | (Arm A) and Study drug in combination with TMZ and | |
| | RT (Arm B). | |
| | Population-level summary measure: DLT rate as a | |
| | function of dose. | |
| | Endpoint: | 2 |
| | Occurrence and severity of TEAE, serious TEAE, | |
| | TEAE leading to death, and TEAE leading to | |
| | treatment discontinuation and/or dose | |
| | modifications, graded according to the NCI- | |
| | CTCAE 5.0 | |
| | Laboratory parameters and related severity | |
| | based on NCI-CTCAE 5.0 | |
| | Occurrence of treatment discontinuations and | |
| | treatment modifications due to TEAEs and | |
| | laboratory abnormalities | |
| | Changes from baseline in parameters of vital | |
| | signs, ECG, ECHO, and KPS | |
| | Population: See Ref. #1 | |
| | Treatment: Study drug in combination with TMZ (Arm | |
| | A) and Study drug in combination with TMZ and RT | |
| | (Arm B). | |
| | Population-level summary measure: | |
| | For occurrence of TEAEs: Percentages of | |
| | participants with at least 1 event. For severity: | |
| | Distribution of severity | |
| | For changes in laboratory values from baseline: | |
| | Proportion of laboratory abnormalities and | |
| | distribution of grading for laboratory abnormalities | |
| | Proportion of treatment discontinuations and | |
| | treatment modifications | |
| | Mean changes from baseline in parameters of vital | |
| | signs, ECG, echocardiogram, and KPS. | |
| Phase 2 | | |
| To assess the efficacy of | Endpoint: OS | 3 |
| Study drug at the RP2D in | Population: Adult participants with a confirmed | |
| combination with TMZ, | diagnosis of GBM IDH-wildtype, Grade 4, who | |
| compared to SOC. | received first-line TMZ-based chemoradiotherapy, | |
| | and have progressed or experienced disease | |
| | recurrence | |
| | Treatment: Study drug in combination with TMZ vs. | |
| | SOC from the external control | |

The Secondary objectives and Estimands (including endpoints) of the clinical trial include:

Secondary Objectives

| | | |
|---|---|---|
| Phase 1 - Arm A | | |
| To determine the PK of | Endpoint: PK profile and PK parameters of Study | 4 |
| Study drug and of its | drug and its metabolite after repeated dosing, | |
| metabolite N³²-desmethyl- | including but not limited to: | |
| Study drug in plasma in | Cₘₐₓ | |
| combination with TMZ. | C_{trough} | |
| | tₘₐₓ | |
| | AUCₗₐₛₜ. | |
| | Population: See Ref. #1 for Phase 1, Arm A | |
| | Treatment: Study drug in combination with TMZ | |
| | In case of no administration of Study drug or event | |
| | such as vomiting or co-administration of a prohibited | |
| | medication, the patient might not be considered for | |
| | PK assessment | |
| | Population-level summary measure: Mean, geometric | |
| | mean, CV of plasma concentrations and PK | |
| | parameters | |
| To determine the PK of TMZ | Endpoint: PK profile and parameters of TMZ after | 5 |
| in plasma in combination with | repeated dosing, including but not limited to: Cₘₐₓ, | |
| Study drug. | AUCₗₐₛₜ, C_{trough} | |
| | Population/In case of/Treatment/Population-level | |
| | summary measures: See Ref. #4 | |
| To investigate preliminary | Endpoint: | 6 |
| antitumor activity of Study | Tumor response according to the RANO criteria: | |
| drug when administered in | BOR, OR, DC | |
| combination with TMZ. | DOR and PFS according to RANO criteria. | |
| | Population: Ref. #1 for Phase 1, Arm A | |
| | Treatment: Study drug in combination with TMZ | |
| | Population-level summary measure: | |
| | Estimate of the ORR and of the DCR | |
| | Distribution of BOR | |
| | Kaplan-Meier median estimates for time-to-event | |
| | endpoints (DOR, PFS). | |
| Phase 1 - Arm B | | |
| To determine the PK of | Endpoint: PK profile and PK parameters of Study | 7 |
| Study drug and of its | drug and its metabolite after first dosing and at | |
| metabolite N³²-desmethyl- | steady-state, including but not limited to: Cₘₐₓ, C_{trough}, | |
| Study drug in plasma in | tₘₐₓ, AUC₂₄ | |
| combination with TMZ and | Population: See Ref. #1 for Phase 1, Arm B | |
| RT. | Treatment: Study drug in combination with TMZ and RT | |
| | In case of/population-level summary measure: See | |
| | Ref. #4 | |
| To determine the PK of TMZ | Endpoint: PK profile and parameters of TMZ after first | 8 |
| in combination with Study | dosing and at steady-state, including but not limited | |
| drug and RT. | to: Cₘₐₓ, AUCₗₐₛₜ, C_{trough} | |
| | Population: See Ref. #1 for Phase 1, Arm B | |
| | Treatment: Study drug in combination with TMZ and RT | |
| | In case of/population-level summary measures: See | |
| | Ref. #4 | |
| To investigate preliminary | Endpoint/Population-level summary measure: See | 9 |
| antitumor activity of Study | Ref. #6 | |
| drug when administered in | Population: See Ref. #1 for Phase 1, Arm B | |
| combination with TMZ and RT. | Treatment: Study drug in combination with TMZ and RT | |
| Phase 2 | | |
| To assess the antitumor | Endpoint: | 10 |
| activity of Study drug when | Tumor response according to RANO criteria: BOR, | |
| administered in combination | OR, DC | |
| with TMZ compared to SOC. | DOR, PFS according to RANO criteria. | |
| | Population: See Ref. #3 | |
| | Treatment: Study drug in combination with TMZ vs. | |
| | SOC from the external control | |
| | Population-level summary measure: | |
| | Estimate of the ORR and of the DCR | |
| | Distribution of BOR | |
| | Kaplan-Meier estimates for time-to-event endpoints | |
| | (DOR, PFS), hazard ratio for PFS | |
| To characterize the safety | Endpoint/Population-level summary measure: See | 11 |
| and tolerability of Study drug | Ref. #2 | |
| when administered in | Population: See Ref. #3 | |
| combination with TMZ, | Treatment: Study drug in combination with TMZ vs. | |
| compared to SOC. | SOC from the external control | |
| To confirm the PK of Study | Endpoint: PK parameters of Study drug and its | 12 |
| drug and of its metabolite | metabolite as applicable, determined using a | |
| N³²-desmethyl-Study drug in | population PK model, including but not limited to: | |
| plasma in combination with | CL/F, apparent V_{d}/F, and ka. | |
| TMZ. | Population: See Ref. #3 | |
| | Treatment: Study drug in combination with TMZ | |
| | In case of: See Ref. #4 | |

The exploratory objectives of the clinical trial include:

Exploratory Objectives

| Phase 1 (dose escalation) & Phase 2 | | |
|---|---|---|
| To investigate antitumor | Endpoint: | 13 |
| activity of Study drug when | Tumor response according to mRANO criteria: | |
| administered in combination | BOR, OR, DC | |
| with TMZ and in combination | DOR and PFS according to mRANO criteria. | |
| with TMZ and RT according | Population: See Ref. #1 for Phase 1 and Ref. #3 for | |
| to mRANO criteria. | Phase 2 | |
| | Treatment: Study drug in combination with TMZ | |
| | Population-level summary measure: See Ref. #6 | |
| To explore brain penetration | Endpoint: | 14 |
| of Study drug, its metabolite | Concentrations of Study drug, its metabolite, and | |
| N³²-desmethyl-Study drug, | TMZ in CSF and in plasma collected at the same | |
| and TMZ. | timepoint | |
| | CSF to plasma ratio of Study drug, its metabolite, | |
| | and TMZ | |
| To explore biomarkers in | Endpoint: Biomarkers (e.g., nucleic acids, | 15 |
| tumor biopsies, blood, and | proteomics) in tumor biopsies, blood, and CSF. | |
| CSF. | | |
| To explore Study drug | Endpoint: PK parameters of Study drug and its | 16 |
| exposure-response | metabolite associated with safety parameters and/or | |
| relationships. | antitumor activity | |
| To assess QoL of | Endpoint: | 17 |
| participants receiving Study | Changes from baseline in FACT-Br score, Brain | |
| drug in combination with | Subscale score, Trial Outcome Index, and FACT- | |
| TMZ in the Phase 2 part of | Br Total Score. | |
| the study. | Time to deterioration (time to first & time to | |
| | confirmed deterioration) in FACT-Br score, Brain | |
| | Subscale score, Trial Outcome Index, and FACT- | |
| | Br Total Score. | |

The study population of this clinical trial is as follows:

### Phase 1:

Arm A: Adult participants (≥18 years old) with a confirmed diagnosis (WHO 2021) of GBM IDH-wildtype, Grade 4, or astrocytoma, IDH-mutant, Grade 3, who received first-line TMZ-based chemoradiotherapy, and have progressed or recurred to a maximum of 2 lines of therapy.
Arm B: Adult participants (≥18 years old) with confirmed, newly diagnosed GBM IDH-wildtype, Grade 4 (WHO 2021).

### Phase 2:

Experimental Arm: Adult participants (≥18 years old) with a confirmed diagnosis (WHO 2021) of GBM IDH-wildtype, Grade 4, who received first-line TMZ-based chemoradiotherapy, and have progressed or experienced disease recurrence.
ECA: Adult patients with a confirmed diagnosis (WHO 2021) of GBM IDH-wildtype, Grade 4, who received first-line TMZ-based chemoradiotherapy, and have progressed or experienced disease recurrence. The ECA will be composed of patients treated with a standard therapy from HCTs.

### Inclusion Criteria:

For both Phase 1 (Arms A and B) and Phase 2, the inclusion criteria include:
(Both) Willing to provide archived or fresh tumor sample, if available. Receipt of tumor sample is not required for the start of study treatment.
(Both) Adequate bone marrow, hepatic, and renal function:
   Absolute neutrophil count (ANC)≥1500/μL, platelets≥100,000/μL, hemoglobin≥9 g/dL or 5.6 mmol/L, and no blood transfusions within the last 2 weeks of study treatment initiation.
   Aspartate aminotransferase (AST) and alanine aminotransferase (ALT)≤2.5×upper limit of normal (ULN) or ≤5×ULN for participants with liver metastases; serum total bilirubin≤1.5×ULN; alkaline phosphatase (ALP)<2.5×ULN or ≤5×ULN for participants with liver metastases.
   Calculated creatinine clearance≥50 mL/minute (min) (as determined by the Chronic kidney disease—Epidemiology Collaboration [CKD EPI] formula).
   Coagulation status: INR or prothrombin time≤1.5×ULN; activated partial thromboplastin time (aPTT)≤1.5×ULN.
(Both) Life expectancy of at least 3 months in the best judgment of the Investigator.
(Both) Participants receiving corticosteroids must be on a stable or decreasing dose of ≤4 mg daily dexamethasone (or ≤25 mg prednisone) for the 7 days prior to the start of study treatment.
(Both) Participants with seizures must be adequately controlled on a stable regimen of anti-epileptic drugs.
   Note: Anticonvulsants with enzyme inhibition or induction properties are prohibited.
(Both) Measurable or non-measurable disease as per RANO criteria by Gd-based contrast-enhanced brain magnetic resonance imaging (MRI).
(Both) Willing to practice highly effective methods of contraception.

### Additional Specific Inclusion Criteria for Phase 1 Arm A:

(A) Participants must have one of the following histopathologically proven diagnoses (WHO 2021):
   GBM IDH-wildtype Grade 4, which may include secondary GBMs (i.e., those that progress from low-grade gliomas)
   Astrocytoma, IDH-mutant, Grade 3
(A) A maximum of 2 prior treatment lines of which first-line must be treatment with TMZ-based chemoradiotherapy (TMZ concomitantly with RT).
(A) Documented disease recurrence or progression by diagnostic biopsy or Gd-based contrast-enhanced brain MRI as per RANO criteria. Progressive disease (PD) to the latest treatment regimen is required. A subsequent brain MRI must be obtained within 10 days prior to the start of study treatment, unless the start of study treatment is within 28 days after the diagnostic brain MRI.
(A) Surgery following the first disease progression will be allowed. A post-operative brain MRI must be available within 72 hours following surgery to avoid interpretation of post-operative changes as residual enhancing disease. A subsequent brain MRI documenting disease progression as per RANO criteria must be obtained within 10 days prior to the start of study treatment. Surgery must have been performed at least 2 weeks before initiation of study treatment and participants should have recovered.
(A) Karnofsky Performance Status (KPS)≥60.

### Additional Specific Inclusion Criteria for Phase 1 Arm B:

(B) Participants must have a new, histopathologically proven diagnosis (WHO 2021) of GBM, IDH-wildtype, Grade 4, which may include secondary GBMs (i.e., those that progress from low-grade gliomas), if the prior treatment included surgery only. The histological diagnosis may be obtained either from a brain biopsy or from a surgical resection.
(B) Study treatment initiation is planned for ≥2 weeks after the diagnostic biopsy or within 3 to 7 weeks after maximal surgical resection (subtotal resection, or gross total resection).
(B) For participants who have undergone tumor resection, a post-operative brain MRI performed within 72 hours is recommended. A subsequent brain MRI must be obtained within 10 days prior to the start of study treatment.
(B) For participants who have undergone diagnostic biopsy only, a subsequent brain MRI must be obtained 10 days prior to the start of study treatment, unless the start of study treatment is within 28 days after the diagnostic brain MRI.
(B) KPS≥70.

### Additional Specific Inclusion Criteria for Phase 2:

(Phase 2) Participants must have a histopathologically proven diagnosis (WHO 2021) of GBM, IDH-wildtype, Grade 4.
(Phase 2) Prior first-line treatment with TMZ-based chemoradiotherapy (TMZ concomitantly with RT), i.e. maximum of 1 prior line of treatment.
(Phase 2) Documented first disease recurrence or progression by diagnostic biopsy or Gd-based contrast-enhanced brain MRI as per RANO criteria. A subsequent brain MRI must be obtained within 10 days prior to the start of study treatment.
(Phase 2) KPS≥60.

### Exclusion Criteria:

For both Phase 1 (Arms A and B) and Phase 2, the exclusion criteria include:
(Both) History of other malignancies requiring active treatment in the last 2 years prior to the first dose of study treatment except for superficial bladder cancers, adequately treated low-risk prostate cancer under active surveillance, ductal carcinoma in situ or other carcinomas in situ, and non-melanoma skin cancers (basal cell/squamous cell skin cancer) that have been treated with curative intent.
(Both) Current use of an investigational agent.
(Both) Known contraindication to undergoing Gd-based contrast-enhanced MRI.
(Both) Major surgery for other diseases within 4 weeks prior to the first dose of study treatment.
(Both) History of myocardial infarction or stroke within 6 months, congestive heart failure greater than New York Heart Association (NYHA) class II, unstable angina pectoris, unexplained recurrent syncope, cardiac arrhythmia requiring treatment or known family history of sudden death from cardiac-related causes before the age of 50, or any cardiotoxicity experienced after previous chemotherapy.
(Both) LVEF below 55%.
(Both) Baseline QTcF interval>450 ms, history of congenital long QT syndrome, or clinically significant conduction abnormality, or any conduction abnormality that may increase the risk of TdP or QTc interval prolongation.
(Both) Concomitant use of a drug with a known risk of QTc prolongation/TdP. If such a drug has been used by the participant, a wash-out period of at least 5 half-lives of the drug must occur before first administration of study treatment.
(Both) Concomitant use of a drug or herbal product that is an inhibitor or inducer of CYP3A4, a strong inhibitor of CYP2D6, or other drugs (such as proton pump inhibitors, H2 receptor antagonists, etc.) mentioned in the prohibited medications section of the protocol (Section 6.3.3). If such a drug has been used by the participant, a wash-out period of at least 5 half-lives (4 weeks for irreversible CYP inhibitors and CYP inducers) of the drug must occur before first administration of study treatment.
(Both) Clinically significant gastrointestinal abnormality that would affect the absorption of the drug (e.g., ulcerative diseases, gastrointestinal dysfunction, uncontrolled nausea, vomiting, diarrhea, malabsorption syndrome, major resection of the small bowel or total gastrectomy, or inflammatory bowel disease).
(Both) Any infection requiring the systemic use of an antibiotic or antiviral agent within 1 week prior to start of study treatment.
(Both) Immunization with live or live-attenuated vaccine within 28 days prior to study inclusion.
(Both) Inability or unwillingness to swallow oral medications.
(Both) Exposure to high levels of UV light, for example occupational exposure to sunlight or sunbathing.
(Both) Chemotherapy, monoclonal antibodies/biologics, investigational treatment, or RT with curative intent within 28 days prior to starting study treatment.
(Both) Unresolved AEs or toxicities due to previous treatments >Grade 1, except hemoglobin (not less than 9 g/dL or 5.6 mmol/L). Note: participants with ≤Grade 2 alopecia or endocrinopathies controlled by replacement therapy (e.g., hypothyroidism due to immune checkpoint inhibitors) are exceptions and may qualify for the study.
(Both) Hypersensitivity to Study drug, TMZ, dacarbazine, or any of the excipients found in the formulation for Study drug or TMZ.
(Both) Known history of infection with human immunodeficiency virus (HIV) or positive HIV ½ serology at screening.
(Both) Known active HCV or HBV infection or positive serology at screening defined as:
   HBV screening tests: both HBV sAg and Anti-HepB core IgG.
   Note: A negative HBV DNA test is required for participants who have a positive Anti-HepB core IgG and negative HBsAg test.
   HCV screening tests: both HCV-antibody and positive viral load HCV-ribonucleic acid (RNA) by polymerase chain reaction (PCR)
(Both) Presence of extracranial metastatic, leptomeningeal disease, infratentorial component, or primary spinal tumors.
(Both) Evidence of intracranial or intratumoral hemorrhage by brain MRI. Participants with resolving post-surgical changes, punctate hemorrhage, or hemosiderin are eligible.
(Both) Participants with clinical or imaging signs of herniation.
(Both) Prior exposure to any WEE1 inhibitor.

### Specific Exclusion Criteria for Phase 1 Arm A:

(A) Prior treatment with more than 2 lines of therapy for GBM, IDH-wildtype, Grade 4, or for astrocytoma, IDH-mutant, Grade 3.
Note: Only 1 prior line of systemic therapy is allowed; combination therapy with TMZ and RT with or without subsequent TMZ maintenance treatment is considered as 1 line. Prior surgery, radiation or localized delivery of therapeutic agents (i.e., carmustine-containing wafers [GLIADEL®]) for first recurrence is allowed.
(A) Prior treatment with bevacizumab or with other VEGF inhibitors or VEGF-receptor signaling inhibitors.
(A) Prior TMZ-related hematological event leading to discontinuation of TMZ during the concurrent chemoradiotherapy.

### Specific Exclusion Criteria for Phase 1 Arm B:

(B) Prior radiation, chemotherapy, biological therapy, interstitial brachytherapy, implanted chemotherapy, therapeutics delivered by local injection or convection-enhanced delivery for GBM.
(B) Prior therapy that would result in an overlap of the radiation fields.

### Specific Exclusion Criteria for Phase 2:

(Phase 2) Prior treatment with more than 1 line of systemic therapy for GBM, IDH-wildtype, Grade 4. Combination therapy with TMZ and RT with or without subsequent TMZ maintenance treatment is considered as 1 line.
(Phase 2) Prior treatment with bevacizumab or with other vascular endothelial growth factor (VEGF) inhibitors or VEGF-receptor signaling inhibitors.
(Phase 2) Prior TMZ-related hematological event leading to discontinuation of TMZ during the concurrent chemoradiotherapy.

The investigational products used in this clinical trial are as follows:

The Study Drug is formulated as 20, 30, 60, 100, 130 and 150 mg hard gelatin capsules. The capsules contain 10 mg mini-tablets (excipients are as follows: microcrystalline cellulose, anhydrous lactose, hydroxypropyl cellulose, sodium starch glycolate, anhydrous colloidal silica, and magnesium stearate). Intermediate capsule strengths may be produced to cover alternative dose levels.

Study drug is taken orally on an empty stomach (i.e., participants must be fasting [only water permitted] for at least 4 hours before and 2 hours after intake).

Concomitant RT for Phase 1 Arm B will be delivered using intensity modulated radiation therapy (IMRT) or 3-dimensional (3D)-conformal radiation (conventionally fractionated regimen of 2 Gy per fraction daily, 5 consecutive days per week, total dose 60 Gy over 30 fractions) for 6 weeks. RT will be administered approximately 1 hour after TMZ intake.

TMZ is formulated as capsules with 5, 20, 100, 140, 180, and 250 mg to be administered orally.

In Phase 1 (Arm A), participants will receive TMZ (150 mg/m²/day) orally, once-daily from D1-5, in 28-day cycles. TMZ will be administered approximately 1 hour following Study drug.

In Phase 1 (Arm B), about 30 min to 1 hour following the administration of Study drug, TMZ (75 mg/m²/day) will be given orally, once-daily, concomitantly with RT (only on the first 5 days of each week) for 6 weeks.

In Phase 2, the participants in the Experimental Arm will receive oral, once-daily dosing in 28-day cycles as follows: Study drug at the RP2D on D1-5, D8-10, and D15-17 and TMZ (150 mg/m²/day) on D1-5. TMZ will be administered approximately 1 hour following Study drug.

### Treatment Duration

### Phase 1 (Dose Escalation):

Study treatment duration for participants in Arm A will be for a maximum of 2 years or until progression of disease, unacceptable toxicity, participant withdrawal, discontinuation as per Investigator's decision, subsequent antineoplastic treatment, end of the study, or death, whichever occurs first. For Arm B, participants are considered to have completed the study treatment period if they have received 6 weeks (42 days) of daily dosing with Study drug with concomitant TMZ and RT. It is up to the discretion of the Investigator to initiate a maintenance treatment with TMZ for 5 days (D1-5), repeated every 28 days, with or without the addition of alternating, electric fields (TTFields Optune®) as per the SOC after a 4-to-6-week break following the end of the study treatment. The maintenance treatment with TMZ occurs after the study treatment period.

### Phase 2:

Treatment duration for participants in Phase 2 will be for a maximum of 2 years or until progression of disease, unacceptable toxicity, participant withdrawal, discontinuation as per Investigator's decision, end of the study, or death, whichever occurs first.

It is estimated that up to 89 evaluable participants will be enrolled in this study.

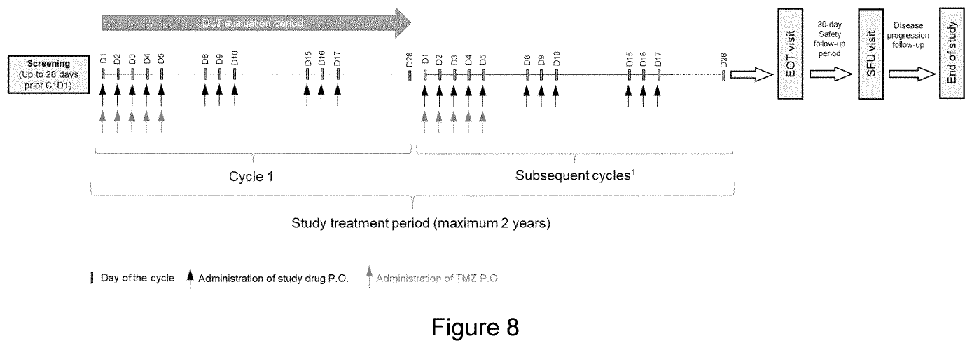

FIGS. 8, 9 and 10 provide study diagrams illustrating Phase 1 Arm A, Phase 1 Arm B and Phase 2 Experimental Arm, respectively.

In these Figures, the following abbreviations are used: AE=Adverse event; C=Cycle; D=Day; DLT=Dose-limiting toxicity; EOS=End of study; EOT=End of treatment; mRANO=Modified Response Assessment in Neuro-Oncology; P.O.=per os; RANO=Response Assessment in Neuro-Oncology; SAE=Serious adverse event; SFU=Safety follow-up; TMZ=Temozolomide; TTFields=Tumor-treating fields; W=Week.

### Specificities for Phase 1 Arm a (FIG. 8):

EOT: 20 days after the last dose of study treatment.

SFU: After the safety follow-up and until EOS for the participant, all SAEs (regardless of causality assessment) and non-serious AEs considered to be related to study treatment as per investigator assessment will be collected.

¹Subsequent cycles will be for a maximum of 2 years or until progression of disease documented by RANO and confirmed by mRANO, unacceptable toxicity, participant withdrawal, discontinuation as per investigator's decision, subsequent antineoplastic treatment, the end of the study, or death, whichever occurs first.

A cycle is defined as 28 days.

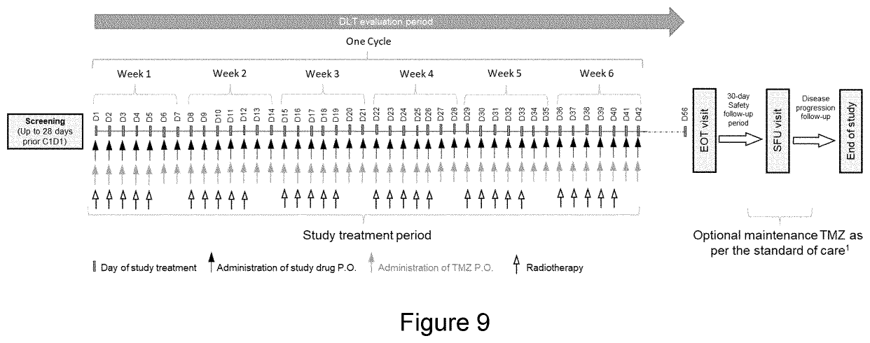

### Specificities for Phase 1 Arm B (FIG. 9):

EOT: 20 days after the last dose of study treatment.

SFU: After the safety follow-up and until EOS for the participant, all SAEs (regardless of causality assessment) and non-serious AEs considered to be related to study treatment as per investigator assessment will be collected.

¹Following end of study treatment and after a 4-to-6 week rest period, maintenance treatment with TMZ for 5 days (D1-5), repeated every 28 days, with or without the addition of alternating, electric, tumor treating fields (TTFields) (Optune®) therapy may be administered at the discretion of the investigator as per the standard of care.

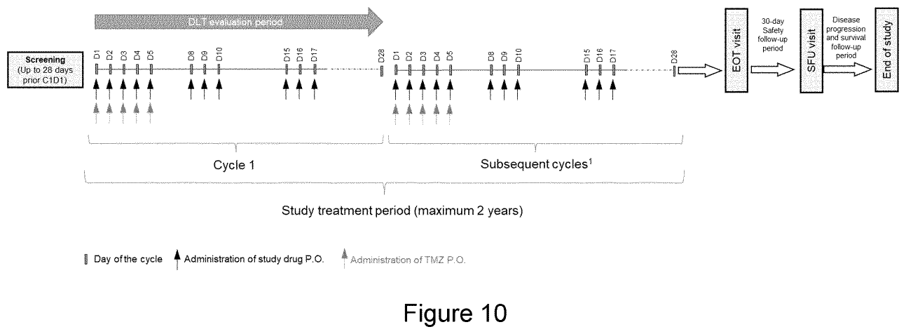

### Specificities for Phase 2 Experimental Arm (FIG. 10):

EOT: 20 days after the last dose of study treatment.

SFU: After the safety follow-up and until EOS for the participant, all SAEs (regardless of causality assessment) and non-serious AEs considered to be related to study treatment as per investigator assessment will be collected.

¹Subsequent cycles will be for a maximum of 2 years or until progression of disease documented by RANO and confirmed by mRANO, unacceptable toxicity, participant withdrawal, discontinuation as per investigator's decision, subsequent antineoplastic treatment, the end of the study, or death, whichever occurs first.

A cycle is defined as 28 days.

### Example 10: Phase 1/2 Clinical Trial Involving Compound of Formula (I) (Referred Herein to as the Study Drug)

A clinical trial as described in Example 9 above will be conducted, which is a Phase 1/2, multi-center, open-label, non-randomized, controlled study in adult participants with glioma, with a new Phase 1 Arm C (dose escalation) as follows.

The study population of this arm C is adult participants with confirmed, newly diagnosed GBM IDH-wildtype, Grade 4 (WHO 2021). The participants will receive continuous, daily doses of TMZ (75 mg/m²/day, oral, once-daily dosing) for 6 weeks with concomitant administration of radiotherapy (RT; conventionally fractionated regimen of 2 Gy per fraction daily, 5 consecutive days per week, total dose 60 Gy over 30 fractions).

The participants will also receive oral, once-daily dosing of Study Drug (escalating doses) for 6 weeks, according to a schedule selected from the following options:
Study drug (escalating doses) on Days (D) 1-3 of each week; or
Study drug (escalating doses) on Days (D) 1-5 of each week.

The starting dose of Study drug is based on available data from the dose levels determined to be safe in the ongoing phase 1 studies, namely the 101-study involving the Study Drug in combination with carboplatin, the 102-study involving the Study Drug in monotherapy, and preliminary data from the study described in Example 9. Such starting dose may be 60 mg or 90 mg, and dose reductions down to 30 mg may be considered, if needed.

In case of unacceptable toxicity observed in the above Study Drug schedules for Arm C, one or more holiday periods (of one week each) may be introduced within the 6-week schedule, for Study Drug only.

Following a 4-to-6-week rest period after the end of the study treatment, a maintenance treatment with TMZ for 5 days (D1-5), repeated every 28 days, with or without the addition of alternating, electric, tumor-treating fields (TTFields) (Optune®) therapy may be administered at the discretion of the Investigator as per the standard of care (SOC).

### Example 11: Compound of Formula (I) (Study Drug) is Present in CSF, Showing that it Effectively Crosses the Blood Brain Barrier and Distributes into the Brain in Glioma Patients

In the clinical trial described in Example 9, cerebrospinal fluid (CSF) stabilized with formic acid 0.5% and matching plasma samples collected on K2 EDTA were drawn from 3 patients of Arm A on day 11 of cycle 1 (i.e. 2 patients at 150 mg and 1 patient at 200 mg of Study drug) and 1 patient of Arm B on Week 3 day 1 (i.e. Day 15 after daily doses of Study drug at 90 mg).

Study drug (parent, active moiety) and its active metabolite N³²-desmethyl-Study drug were measured using LC-MS/MS methods. Results were provided in ng per mL of plasma or CSF, and then converted to nmol/L using the molecular weights of the Study drug (511.45 g/mol) and N³²-desmethyl-Study drug (497.42 g/mol).

In plasma, the active metabolite represents about 10% of the Study drug parent and both compounds are moderately bound to plasma proteins: 91.7% for Study drug parent and 87.1% for N³²-desmethyl-Study drug. Thus, the unbound (or free fraction) of the Study Drug and the N³²-desmethyl-Study drug are calculated.

In CSF, the total protein concentration varies between 0.2% and 0.5% of the total protein concentration of blood (H. Reiber, Clinica Chimica Acta, 2001; Schilde L M et al., PLoS ONE, 2018), therefore the concentration measured in CSF essentially corresponds to the free fraction of the compounds.

For each of the 4 patients, the ratio CSF/plasma of a given compound (Study drug parent or metabolite) was calculated using the CSF concentration of said compound (in nmol/L) and the unbound or free fraction of said compound in plasma (in nmol/L). Raw data and results are presented in Tables 5 and 6.

TABLE 5

| | | | | Raw data | | Raw data | Unbound |
|---|---|---|---|---|---|---|---|
| | | | Dose | concentration | CSF | concentration | plasma |
| Active | | | level | in CSF | concentration | in plasma | concentration |
| Moiety | Patient | Arm | (mg) | (ng/mL) | (nmol/L) | (ng/mL) | (nmol/L) |
| Study drug | 1 | A | 150 | 10.2 | 19.94 | 346 | 56.29 |
| | 2 | A | 150 | 2.43 | 4.75 | 88.2 | 14.35 |
| | 3 | A | 200 | 10.1 | 19.75 | 322 | 52.38 |
| | 4 | B | 90 | 10.9 | 21.31 | 309 | 50.27 |
| N³²- | 1 | A | 150 | 0.114 | 0.23 | 17.7 | 4.59 |
| desmethyl- | 2 | A | 150 | 0.128 | 0.26 | 15.6 | 4.05 |
| Study drug | 3 | A | 200 | 0.154 | 0.31 | 23.2 | 6.02 |
| | 4 | B | 90 | 0.302 | 0.61 | 42.8 | 11.10 |
| Study drug + | 1 | A | 150 | NA | 20.17 | NA | 60.88 |
| N³²- | 2 | A | 150 | NA | 5.01 | NA | 18.39 |
| desmethyl- | 3 | A | 200 | NA | 20.06 | NA | 58.40 |
| Study drug | 4 | B | 90 | NA | 21.92 | NA | 61.37 |

TABLE 6

| | | | | CSF to “unbound |
|---|---|---|---|---|
| Active Moiety | Patient | Arm | Dose level (mg) | plasma” ratio |
| Study drug | 1 | A | 150 | 0.35 |
| | 2 | A | 150 | 0.33 |
| | 3 | A | 200 | 0.38 |
| | 4 | B | 90 | 0.42 |
| N³²- | 1 | A | 150 | 0.05 |
| desmethyl- | 2 | A | 150 | 0.06 |
| Study drug | 3 | A | 200 | 0.05 |
| | 4 | B | 90 | 0.05 |
| Study drug + | 1 | A | 150 | 0.33 |
| N³²- | 2 | A | 150 | 0.27 |
| desmethyl- | 3 | A | 200 | 0.34 |
| Study drug | 4 | B | 90 | 0.36 |

The CSF/plasma ratio for the sum of active moieties (Study drug and N³²-desmethyl-study drug) is about 1/3, demonstrating relatively high levels in CSF and therefore good drug penetration in patient brain.

While CSF/plasma ratio for N³²-desmethyl-Study drug concentration is about 5%, the ratio for parent drug is 33 to 42%, which demonstrates a particularly high propensity for brain penetration of the Study drug.

1. A method of treating glioma in a patient in need thereof, the method comprising administering to the patient a compound of formula (I) 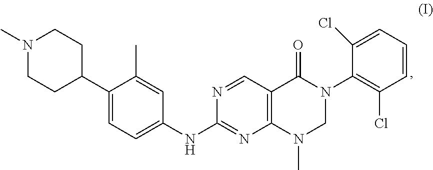 [SMILES: Cc1cc(Nc2ncc3c(n2)N(C)CN(c2c(Cl)cccc2Cl)C3=O)ccc1C1CCN(C)CC1]
or a pharmaceutically acceptable salt thereof.

2. The method of claim 1, wherein the compound of formula (I) is administered in combination with temozolomide (TMZ) and/or radiotherapy.

3. The method of claim 1, wherein the glioma is glioblastoma, astrocytoma or oligodendroglioma.

4. The method of claim 1, wherein the glioma is a high-grade glioma (Grade 3 or 4).

5. The method of claim 1, wherein the compound of formula (I) is administered orally,
(a) at an intermittent schedule, selected from any one of (i) to (vi)
i. on days 1 to 5, 8 to 10 and 15 to 17 of a 28-day cycle,
ii. on days 1 to 5, 8 to 12 and 15 to 19 of a 28-day cycle,
iii. on days 1 to 5, 8 to 12, 15 to 19 and 22-26 of a 28-day cycle,
iv. on days 1 to 3 of each week, for 6 or 7 weeks,
v. on days 1 to 5 of each week, for 6 or 7 weeks, or
vi. on days 1 to 4 of each week, for 6 or 7 weeks, or
(b) at a continuous schedule, selected from:
i. daily for 6 weeks,
ii. daily for 7 weeks, or
iii. daily for 42 to 49 days.

6. The method of claim 1, wherein the compound of formula (I) is administered over 1, 2, 3, 4, 5, 6 or more 28-day cycles.

7. The method of claim 1, wherein the compound of formula (I) is administered
i. at a dose ranging from about 30 to about 1000 mg per treatment day, or
ii. at a dose ranging from about 30 to about 720 mg per treatment day, and/or
iii. as a single dose on a treatment day, and/or
iv. at approximately the same time on each treatment day.

8. The method of claim 1, wherein the compound of formula (I) is used in combination with temozolomide, and temozolomide is administered
i. at a dose of 50-300 mg/m²/day on days 1 to 5 of a 28-day cycle,
ii. at a dose of 50-250 mg/m²/day on days 1 to 5 of a 28-day cycle,
iii. at a dose of 50-200 mg/m²/day on days 1 to 5 of a 28-day cycle,
iv. at a dose of 150 mg/m²/day on days 1 to 5 of a 28-day cycle,
v. at a dose of 200 mg/m²/day on days 1 to 5 of a 28-day cycle,
vi. at a dose of 50-300 mg/m²/day, daily for 6 weeks, daily for 7 weeks or daily for 42 to 49 days,
vii. 50-250 mg/m²/day, daily for 6 weeks, daily for 7 weeks or daily for 42 to 49 days,
viii. 50-200 mg/m²/day, daily for 6 weeks, daily for 7 weeks or daily for 42 to 49 days, or
ix. at a dose of 75 mg/m²/day, daily for 6 weeks, daily for 7 weeks or daily for 42 to 49 days.

9. The method of claim 1, wherein the compound of formula (I) is administered in combination with temozolomide, and on a WEE1 inhibitor treatment day, if temozolomide is administered on said WEE1 inhibitor treatment day, administration of the compound of formula (I) precedes administration of temozolomide, optionally by a period of about 30 min to 4 h.

10. The method of claim 1, wherein the compound of formula (I) is administered in combination with temozolomide, and on a WEE1 inhibitor treatment day, if temozolomide is administered on said WEE1 inhibitor treatment day, temozolomide administration is concomitant to administration of the compound of formula (I).

11. The method of claim 1, wherein said compound of formula (I) is administered in combination with temozolomide and radiotherapy.

12. The method of claim 11, wherein the compound of formula (I) is administered orally,
(a) at an intermittent schedule selected from:
i. on days 1 to 3 of each week, for 6 or 7 weeks,
ii. on days 1 to 5 of each week, for 6 or 7 weeks, or
iii. on days 1 to 4 of each week, for 6 or 7 weeks, or
(b) at a continuous schedule,
(c) daily for 6 weeks,
(d) daily for 7 weeks, or
(e) daily for 42 to 49 days, and
wherein temozolomide is administered at
(x) a dose of 50-300 mg/m²/day, daily for 6 weeks, daily for 7 weeks, or daily for 42 to 49 days,
(y) 75 mg/m²/day, daily for 6 weeks, daily for 7 weeks, or daily for 42 to 49 days.

13. The method of claim 11, wherein the radiotherapy is fractionated radiotherapy and is administered
i. in a regimen of 1 Gy to 100 Gy, divided over 5 consecutive days per week until a total radiation dose has been administered, or in equal fractions over a period of 6 weeks, 7 weeks or 42-49 days,
ii. in a regimen of 2 Gy to 80 Gy, divided over 5 consecutive days per week until a total radiation dose has been administered, or in equal fractions over a period of 6 weeks, 7 weeks or 42-49 days,
iii. in a regimen of 10 Gy to 60 Gy, divided over 5 consecutive days per week until a total radiation dose has been administered, or in equal fractions over a period of 6 weeks, 7 weeks or 42-49 days,
iv. in a regimen of 60 Gy divided over 5 consecutive days per week until a total radiation dose has been administered, or in equal fractions over a period of 6 weeks, 7 weeks or 42-49 days,
v. in a regimen of 2 Gy per fraction daily, 5 consecutive days per week for a total of 60 Gy, or
vi. at a total dose of 60 Gy over 30 fractions, over a period of 6 weeks, 7 weeks or 42 to 49 days.

14. The method of claim 1, wherein the glioma is newly diagnosed.

15. The method of claim 1, wherein the glioma has progressed or recurred after initial or prior glioma treatment.

16. The method of claim 15, wherein the initial or prior glioma treatment comprises radiotherapy administration.

17. The method of claim 15, wherein the initial or prior glioma treatment comprises TMZ administration.

18. The method of claim 1, wherein the glioma is glioblastoma.

19. The method of claim 1, wherein the glioma is (IDH)-wildtype as per WHO 2021.

20. The method of claim 1, wherein the glioma is astrocytoma.

21. The method of claim 1, wherein the glioma is IDH-mutant as per WHO 2021.
